(19) United States
(12) Patent Application Publication
(10) Pub. No.: US 2024/0304111 A1
(43) Pub. Date: Sep. 12, 2024

(54) PATIENT SIMULATION TRAINING SYSTEM FOR A BREATHING ASSISTANCE OR RESPIRATORY APPARATUS

(71) Applicant: Fisher & Paykel Healthcare Limited, Auckland (NZ)

(72) Inventors: Hazel Sarah GLASGOW, Auckland (NZ); Anton Kim GULLEY, Auckland (NZ); Jin Kyu LIM, Auckland (NZ); Philip Ian ROWE, Auckland (NZ); Yunong XU, Auckland (NZ)

(21) Appl. No.: 18/569,138

(22) PCT Filed: Jun. 15, 2022
(86) PCT No.: PCT/IB2022/055524

§ 371 Date: Dec. 11, 2023

Related U.S. Application Data
(60) Provisional application No. 63/210,932, filed on Jun. 15, 2021.

(30) Foreign Application Priority Data
Aug. 24, 2021 (AU) 2021221509

(51) Int. Cl. G09B 23/28; A61M 16/00; G09B 5/02; G16H 20/40; G16H 50/50

(52) U.S. Cl. CPC G09B 23/28; A61M 16/026; G09B 5/02; G16H 20/40; G16H 50/50

(57) ABSTRACT

In learning to use a respiratory apparatus, operators need to be able to train in a safe and practical way. This disclosure includes systems and methods on how to train operators through the use of simulation, which does not require the respiratory apparatus to be used by a live patient or real oxygen source to be connected to the respiratory apparatus. The simulation of a patient thus can provide a safe training system in a constrained environment to train operators without the need to train on actual patients or use O₂. This allows operators to train in a safe manner by avoiding injury to patients or potential mishaps with O₂ and its storage.

### TECHNICAL FIELD

The present disclosure relates to training for control of components of breathing assistance or respiratory apparatuses.

### BACKGROUND ART

Breathing assistance or respiratory apparatuses are used to deliver a flow of gas(es) to patients in various environments (such as hospital, medical facility, residential care, or home environments). A breathing assistance or respiratory apparatus (e.g. a respiratory apparatus) may include an oxygen inlet that enables the respiratory apparatus to deliver supplemental oxygen with the flow of gas(es). A breathing assistance or respiratory apparatus may also (or alternatively) include a humidification apparatus that enables the respiratory apparatus to deliver heated and humidified gases. A breathing assistance or respiratory apparatus may allow adjustment of, and control over, characteristics of the gases flow. These characteristics may include for example flow rate, temperature, gas concentration (such as supplemental oxygen concentration), humidity, and pressure, etc.

Patients suffering from various health conditions and diseases can benefit from breathing assistance (for example respiratory therapy). In at least one form, the respiratory therapy may be oxygen therapy. For example, a patient suffering from chronic obstructive pulmonary disease (COPD), pneumonia, asthma, bronchopulmonary dysplasia, heart failure, cystic fibrosis, sleep apnea, lung disease, trauma to the respiratory system, acute respiratory distress, and/or other conditions or diseases can benefit from respiratory therapy. Similarly, patients receiving pre- and post-operative oxygen delivery can also benefit from respiratory therapy.

### SUMMARY

The present disclose provides a training system for developers and engineers as well as clinical or non-clinical health practitioners (nurses, doctors, respiratory therapists, etc.), which simulates patient physiology and physiological responses to treatment with a breathing assistance or respiratory apparatus. The training system provides a safe environment for operators (hereinafter, any persons operating the respiratory apparatus will be referred to as “operators”) to learn how to use the various features of the breathing assistance apparatus without the need for a patient and/or a real oxygen source to be connected. This is particularly useful where operators are unfamiliar with new technologies or components related to providing breathing assistance to a patient. The present disclosure provides a training platform for both instruction and experimentation.

A training system for a respiratory apparatus according to the present disclosure can include: a respiratory apparatus, and a peripheral device which can stimulate one or more auxiliary device outputs and/or patient respiratory responses. The peripheral device can communicate with the respiratory apparatus and provide the simulated one or more auxiliary device outputs and/or patient respiratory responses.

In some configurations, the peripheral device can be one or more of a tablet, smartphone or personal computer.

In some configurations, the one or more auxiliary devices can include a pulse oximeter device.

In some configurations, the peripheral device can include software configured to model a virtual patient receiving therapy and provide feedback to the respiratory apparatus to simulate measurements of the virtual patient receiving therapy.

In some configurations, the virtual patient can be configured to account for different oxygen requirements of different types of respiratory therapies.

In some configurations, the virtual patient can be configured to account for the different oxygen requirements by adjusting one or more model parameters depending on a therapy mode configured on the respiratory apparatus.

In some configurations, the virtual patient can be configured to model an SpO₂/FiO₂ ratio.

In some configurations, the virtual patient can include a plurality of patient models of different ages that are related by linear factor(s).

In some configurations, the plurality of patient models can include an adult model, a pediatric model, and a neonatal model.

In some configurations, a user interface of the peripheral device can include one or more patient model choice elements configured to allow selection of a predefined patient model based on age.

In some configurations, the virtual patient can be configured to account for different severity statuses.

In some configurations, a user interface of the peripheral device can include one or more patient model choice elements configured to allow selection of a predefined severity status.

In some configurations, the severity statues can include ICU, HDU, and regular ward.

In some configurations, the severity statues can include a respiratory condition, ailment, disease, injury, and/or infection.

In some configurations, the respiratory condition, ailment, disease, injury, and/or infection can include a stage I, II, III, or IV chronic obstructive pulmonary disease (COPD).

In some configurations, the respiratory apparatus can be designed to administer high flow therapy.

In some configurations, the respiratory apparatus can include a flow generator and connection to a supplementary gases supply.

In some configurations, the respiratory apparatus can include a humidifier.

In some configurations, the simulated one or more auxiliary device outputs and/or patient respiratory responses can include information representative of the virtual patient's SpO₂ data.

In some configurations, the virtual patient's SpO₂ data can include plethysmography waveforms.

In some configurations, the simulated one or more auxiliary device outputs and/or patient respiratory responses can include information representative of the virtual patient's respiratory rate.

In some configurations, the simulated one or more auxiliary device outputs and/or patient respiratory responses can include information representative of the virtual patient's minute ventilation, tidal volume, and/or peak inspiratory demand.

In some configurations, the peripheral device can be configured with further instructions to transmit supplementary gas signals that is representative of a supplementary gas being received into the respiratory apparatus.

In some configurations, the peripheral device can be configured with further instructions to continuously transmit the simulated one or more auxiliary device outputs and/or patient respiratory responses and O₂ signals, wherein the simulated one or more auxiliary device outputs and/or patient respiratory responses can be representative of the virtual patient's characteristics, and wherein the O₂ signals can be representative of an O₂ supply.

A peripheral device according to the present disclosure can communicate with a respiratory apparatus to provide simulated outputs of at least one auxiliary device connectable to the respiratory apparatus, the peripheral device including: a first communication interface, wherein the first communication interface can electrically connect the peripheral device to an electrical or electronic communications port of the respiratory apparatus; and a hardware processor operating software that can provide instructions to a hardware processor to: communicate simulated measurement information from the at least one auxiliary device to the electrical or electronic communications port of the respiratory apparatus.

In some configurations, the peripheral device can include software configured to model a virtual patient receiving therapy and communicate feedback to the respiratory apparatus in the form of the simulated measurement information of the virtual patient.

In some configurations, the software can further provide instructions to the hardware processor to adjust a virtual patient parameter configured to alter the simulated measurement information.

In some configurations, the first communication interface can be a connection via a USB port, a Bluetooth interface, or another wireless communication interface.

In some configurations, the peripheral device can be one or more of a tablet, smartphone or personal computer.

In some configurations, the at least one auxiliary device can include a pulse oximeter device.

In some configurations, the simulated measurement information can include information representative of the virtual patient's SpO₂ data.

In some configurations, the virtual patient's SpO₂ data can include plethysmography waveforms.

In some configurations, the hardware processor can be configured with further instructions to simulate the virtual patient such that the respiratory apparatus receives signals that are representative of the virtual patient's response.

In some configurations, the signals that can be representative of the virtual patient's response include signals representative of the virtual patient's respiratory rate.

In some configurations, the signals that are representative of the virtual patient's response can include signals representative of the virtual patient's minute ventilation, tidal volume, and/or peak inspiratory demand.

In some configurations, the hardware processor can be configured with further instructions to transmit supplementary gas signals that are representative of a supplementary gas being received into the respiratory apparatus.

In some configurations, the hardware processor can be configured with further instructions to continuously transmit virtual patient response signals and O₂ signals, wherein the virtual patient response signals can be representative of the virtual patient's characteristics and wherein the O₂ signals can be representative of an O₂ supply.

In some configurations, the peripheral device can further include a second communication interface, wherein the second communication interface can electrically connect the peripheral device to another auxiliary device.

In some configurations, the second communication interface can be a connection via a USB port, a Bluetooth interface, or another wireless communication interface.

In some configurations, the another auxiliary device connected to the peripheral device via the second communication interface can be an artificial lung.

A respiratory apparatus training system according to the present disclosure can include a respiratory apparatus and a peripheral device. The respiratory apparatus can include: a valve configured to control an amount of supplementary gases entering the respiratory apparatus; a flow generator configured to generate a flow of one or more gases, wherein the one or more gases can include the supplementary gases or the respiratory apparatus can be configured to simulate administration of the supplementary gases in response to one or more signals from a peripheral device simulating a virtual patient receiving therapy from the respiratory apparatus; an apparatus user interface, wherein the respiratory apparatus can be configured to receive operating parameters and/or operational settings based on input received via the apparatus user interface; an apparatus communications interface configured to receive and transmit the one or more signals from the peripheral device; and an apparatus controller, wherein the apparatus controller can be configured to control the flow generator and the valve and transmit signals to the apparatus user interface, wherein the respiratory apparatus can be configured to operate based on the received operating parameters and/or operational settings and the one or more signals from the peripheral device. The peripheral device can include: a peripheral device controller; a peripheral device user interface; and a peripheral device communications interface to receive and transmit the one or more signals to the respiratory apparatus, wherein the one or more signals to the respiratory apparatus can further indicate a plurality of parameters of the virtual patient in response to therapy administered by the respiratory therapy device, the peripheral device user interface configured to allow the plurality of parameters and/or characteristics of the virtual patients to be modified.

In some configurations, the supplementary gases can include oxygen.

In some configurations, the respiratory apparatus can be configured to adjust a FdO₂ value of the flow of one or more gases.

In some configurations, the apparatus controller can be configured to control the valve so as to adjust the FdO₂ value.

In some configurations, the apparatus controller can be configured to adjust the FdO₂ value in response to the one or more signals from the peripheral device including a selection of a high pressure oxygen source.

In some configurations, the plurality of parameters can include the virtual patient's SpO₂.

In some configurations, the plurality of parameters can include a supplementary gases source.

In some configurations, the characteristics of the virtual patient can include age, severity status, and/or ward type.

In some configurations, settings of the peripheral device can be configured to be modified by a user to change the one or more signals.

In some configurations, the training system can be used for training an operator of the respiratory apparatus.

In some configurations, the respiratory apparatus can further include a supplementary gas source port in communication with the valve.

In some configurations, the peripheral device can further include a non-volatile memory.

A training system for training operators in the use of a respiratory support device according to the present disclosure can include a respiratory apparatus and a peripheral device. The respiratory apparatus can include: a flow generator configured to generate a flow of one or more gases; an apparatus user interface, wherein the respiratory apparatus can be configured to receive operating parameters and/or operational settings based on inputs received at the apparatus user interface; an apparatus communications interface to receive from and transmit signals to another device in electrical communication with the respiratory apparatus. The peripheral device can include: a peripheral device controller; a peripheral device user interface; and a peripheral device communications interface to receive from and transmit signals to another device in electrical communication with the peripheral device, wherein the peripheral device can be configured to communicate with the respiratory apparatus to transmit simulated signals including a signal representative of a supplementary gases and a signal representative of a virtual patient response; wherein the training system can be configured to allow an operator to learn operation of the respiratory apparatus based on the simulated signals from the peripheral device.

In some configurations, the peripheral device user interface can be configured to allow the operator to modify the signal representative of the supplementary gases and/or the signal representative of the virtual patient response.

In some configurations, the apparatus user interface can be configured to allow the operator to change the operating parameters and/or operational settings.

In some configurations, the respiratory apparatus can be configured to have a training mode, wherein the apparatus user interface can be configured to allow the operator to enter the training mode, wherein the training mode can allow the respiratory apparatus to receive the simulated signals from the peripheral device and execute the operational parameters and/or operational settings that are set by the operator.

In some configurations, the respiratory apparatus can be a high flow apparatus that provides high flow therapy.

In some configurations, the respiratory apparatus can further include a supplementary gases source port, a valve in communication with the supplementary gases port to control an amount of supplementary gases entering the respiratory apparatus.

In some configurations, the respiratory apparatus can further include a humidifier and a heated breathing tube.

In some configurations the respiratory apparatus comprises a controller that is configured to determine if a chamber has been removed and the controller is configured to allow operation within the training system when the chamber has been determined as removed. This functions as a safety mechanism wherein if the chamber is detected as being in an operative position i.e. not removed, then this is an indication that the respiratory support device is potentially being used by a patient and therefore should not be used as part of the training system.

In some configurations, the respiratory apparatus is configured to determine a chamber off condition (i.e. humidification chamber removed), wherein the apparatus is configured to perform a first leak determination method that comprises comparing measured pressure at a current flow rate, determining if the measured pressure is indicative of a chamber off condition, if there is no leak condition detected, then the apparatus is configured to disable or lock communication with the peripheral device and if there is a leak condition detected, the apparatus is configured to enable communication with the peripheral device.

The apparatus is configured to perform a second leak determination if the first leak determination results in a possible leak condition being detected, wherein the second leak determination comprises increasing the flow rate in set increments (or increasing motor speed by set increments) and after each increase checking the measured pressure for the current flow rate and determining if the measured pressure is below a leak threshold. If the measured pressure is below a leak threshold, the second leak determination provides a confirmation of a chamber leak condition i.e. a chamber off condition. The respiratory apparatus controller is configured to allow communication

### BRIEF DESCRIPTION OF THE DRAWINGS

Specific embodiments and modifications thereof will become apparent to those skilled in the art from the detailed description herein having reference to the figures that follow, of which:

FIG. 1 shows in schematic form an example breathing assistance apparatus.

FIGS. 2, 2A and 2B show perspective views of example breathing assistance apparatuses.

FIG. 2C illustrates a left front perspective partial cutaway view showing a valve module and a filter module.

FIG. 3 illustrates an example graphical user interface display of an example breathing assistance apparatus.

FIG. 4 is a schematic diagram of an example training system.

FIG. 5 is a schematic diagram of an example training system.

FIGS. 6A, 6B, 6C, and 6D illustrate an example graphical user interface display on a peripheral device of a training system.

FIG. 7 illustrates an example graph generated by a graphical user interface display on a peripheral device of a training system.

FIG. 8 illustrates a Smith predictor being utilized with the PID controller in a closed loop oxygen control system.

FIG. 9 illustrates a summary flow diagram of an example leak detection algorithm.

### DETAILED DESCRIPTION

A breathing assistance or respiratory apparatus can provide therapy to a user. The therapy can include, for example, any one or any combination of: Nasal High Flow (NHF) therapy, Continuous Positive Airway Pressure (CPAP) therapy, Non-Invasive Ventilation (NIV), and Bubble Continuous Positive Airway Pressure (BCPAP) therapy.

The term breathing assistance apparatus may be used interchangeably with respiratory apparatus.

The term breathing assistance system may be used interchangeably with respiratory system.

CPAP therapy may comprise providing gases to a user at a continuous positive pressure (and optionally one or more therapy parameters as disclosed herein in more detail below.)

BCPAP therapy may comprise providing gases to a user (for example, a neonatal patient) at a therapy flow rate (and optionally one or more therapy parameters as disclosed herein in more detail below.)

Non-invasive ventilation (NIV) (for example, Bi-level PAP therapy also known as BiPAP) may comprise providing gases to a user at a therapy inspiratory positive airway pressure (IPAP) and expiratory positive airway pressure (EPAP) (and optionally one or more therapy parameters as disclosed herein in more detail below.)

High flow therapy as discussed herein is intended to be given its typical ordinary meaning as understood by a person of skill in the art and generally refers to a breathing assistance apparatus delivering a targeted flow of humidified respiratory gases via an intentionally unsealed patient interface. Additional details about high flow therapy will be described in greater detail below.

In the context of receiving therapy the user is a patient. However, in the context of interacting with the apparatus (for example interacting with a user interface) the user can be one or more of a patient, healthcare professional (for example a clinician), or anyone else interested in using the apparatus.

As used herein, a “gases flow” or a “flow of gases” can refer to any flow of gases that may be provided by the breathing assistance apparatus, such as a flow of ambient air, a flow comprising substantially 100% oxygen, a flow comprising some combination of ambient air and oxygen, and/or the like.

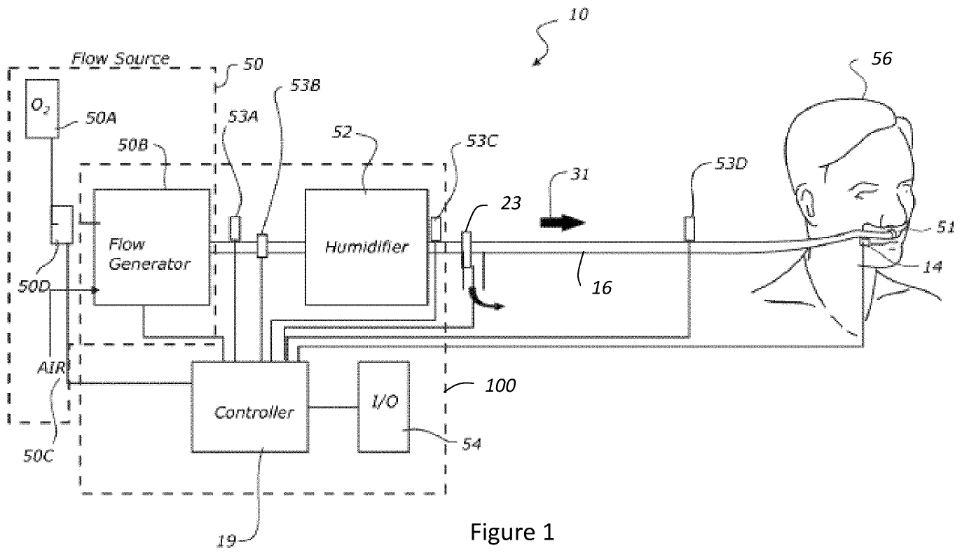

A schematic representation of the example respiratory apparatus 10 is provided in FIG. 1. The respiratory apparatus 10 (or “respiratory system”) can include a flow source 50 for providing a high flow gases 31, such as air, oxygen, air blended with oxygen, or a mix of air and/or oxygen and one or more other gases. Alternatively, the breathing assistance apparatus 10 can have a connection for coupling to a flow source. As such, the flow source might be considered to form part of the apparatus or be separate to it, depending on context, or even part of the flow source may form part of the apparatus, and part of the flow source may fall outside of the apparatus. In short, depending on the configuration (e.g., some components may be optional), the respiratory apparatus or system can include a combination of components selected from the following: a flow source; a humidifier for humidifying the gas-flow; a conduit (e.g. dry line or heated breathing tube); a patient interface; a non-return valve; a filter, etc. During normal operation, the gas(es) flow from the flow source to an inlet of the humidifier, and then from an outlet of the humidifier (for example, an outlet of a humidification chamber, also referred to herein as a “chamber outlet”), through a delivery conduit (for example, heated breathing tube) to a patient interface.

The respiratory apparatus or system 10 will now be described in more detail. The flow source 50 could include an in-wall supply of oxygen, a tank of oxygen, a tank of other gas and/or a high flow apparatus with a flow generator 50B. FIG. 1 shows a non-limiting example the flow source 50 including a flow generator 50B. The flow source 50 can optionally include an air inlet 50C. Additionally or alternatively, the flow source 50 can optionally include a connection to an O₂ source (such as O₂ tank or O₂ generator) 50A via a gas flow control 50D. The gas flow control 50D can be for example, a shut off valve, a regulator, and/or otherwise. The flow generator 50B can control flows delivered to the patient 56 using one or more valve, or optionally the flow generator 50B can comprise a blower. The flow source could be one or a combination of a flow generator 50B, O₂ source 50A, air source 50C as described. The flow source 50 is shown as part of the apparatus 10. However, in the case of an external oxygen tank or in-wall source, the flow source 50 may be considered a separate component, in which case the apparatus 10 has a connection port to connect to such flow source. The flow source can provide a flow of gases that can be delivered to a patient via a delivery conduit 16, and patient interface 51. The flow of gases provided to the patient 56 may be a high flow of gases (as will be discussed in greater detail below).

The patient interface 51 of the respiratory apparatus 10 may be an unsealed (non-sealing) interface (for example when used in high flow therapy, that is when a high flow of gases is delivered to the patient 56) or a sealed (sealing) interface (for example when used in CPAP). Non-limiting examples of an unsealed patient interface 51 include a non-sealing nasal cannula. Non-limiting examples of a sealed patient interface 51 includes a nasal mask, full face mask, or nasal pillows. The patient interface 51 may be a non-sealing patient interface which would help to prevent barotrauma, which can include tissue damage to the lungs or other organs of the respiratory system due to difference in pressure relative to the atmosphere, particularly when high flow therapy is provided to the patient. The patient interface 51 may be a sealing mask that seals with the patient's nose and/or mouth. The patient interface may be a nasal cannula with a manifold and nasal prongs, and/or a face mask, and/or a nasal pillows mask, and/or a nasal mask, and/or a tracheostomy interface, or any other suitable type of patient interface. The flow source could provide a base gases flow rate of between, e.g. 0.5 litres/min and 375 litres/min, or any range within that range, or even ranges with higher or lower limits. Details of the ranges and nature of flow rates will be provided in more detail below.

The humidifier 52 can be between the flow source 50 and the patient 56 to provide humidification of the delivered gas. In some configurations the humidifier may be optional, or it may be preferred due to the advantages of humidified gases helping to maintain the condition of the airways.

One or more sensors 53A, 53B, 53C, 53D such as flow, oxygen fraction, pressure, humidity, temperature or other sensors can be placed throughout the apparatus or system 10 and/or at, on or near the patient 56. For example, one or more sensors 53A, 53B can be located downstream of the flow source 50 and/or upstream of the humidifier 52. As another example, one sensor 53C may be located downstream of the humidifier 52, such as at the outlet of the humidifier 52. As yet another example, one sensor 53D can be located along the delivery conduit 16. Alternatively or additionally, sensors from which such parameters can be derived could be used. In addition, or alternatively, the sensors 53A-53D can be one or more physiological sensors for sensing patient physiological parameters such as, heart rate, oxygen saturation, partial pressure of oxygen in the blood, respiratory rate, partial pressure of CO₂ in the blood. Alternatively, or additionally, sensors from which such parameters can be derived could be used. Other patient sensors could comprise EEG sensors, torso bands to detect breathing, and any other suitable sensors. One or more of the sensors might form part of the apparatus, or be external thereto, with the apparatus having inputs for any external sensors. The sensors can be coupled to or send their output to a controller 19 of the respiratory apparatus 10.

The respiratory system 10 can include a sensor 14 for measuring the oxygen fraction of air the patient inspires. The sensor 14 can be placed on the patient interface 51, to measure or otherwise determine the fraction of oxygen proximate (at/near/close to) the patient's mouth and/or nose. In some configurations, the output from the sensor 14 is sent to the controller 19 to assist control of the respiratory apparatus 10 and to alter operation of the respiratory apparatus 10 accordingly. In some configurations, the sensor 14 can convey measurements of oxygen fraction at the patient mouth and/or nose to a user (who can be the patient and/or any operator of the respiratory apparatus 10), who can input the information to the respiratory apparatus 10/controller 19, for example, via an input/output (I/O) user interface 54 that is in communication with the controller 19. In addition to the sensors 53A-53D described above, the controller 19 is coupled to the flow source 50, humidifier 52 and sensor 14. The controller 19 controls these and other aspects of the respiratory system 10 as described herein. The controller can operate the flow source 50 to provide the delivered flow of gases at a desired flow rate, for example, high enough to meet or exceed the patient's inspiratory demand. The flow rate provided is sufficient that ambient gases are not entrained as the patient inspires.

An optional non-return valve 23 may be provided in the delivery conduit 16. A filter or filters may be provided at the air inlet 50C and/or inlets to the flow generator 50B to filter the incoming gases before the gases are pressurized into a high flow gases 31 by the flow generator 50B.

The breathing assistance or respiratory apparatus 10 could have an integrated or a separate component-based arrangement, generally shown in the dotted box 100 in FIG. 1. In some configurations, the respiratory apparatus or system could be a modular arrangement of components. Furthermore, the respiratory apparatus or system may include only some of the components shown, not necessarily all are essential. Also, the conduit and patient interface do not have to be part of the respiratory system, and could be considered separate. Hereinafter the breathing assistance or respiratory apparatus will be referred to as a breathing assistance apparatus or respiratory system, but the terms should not be considered limiting. Breathing assistance apparatus and respiratory system will be broadly considered herein to comprise anything that provides a flow of gases at a certain flow rate to a patient. Some such apparatus and systems include a detection system that can be used to determine if the flow rate of gases meets inspiratory demand.

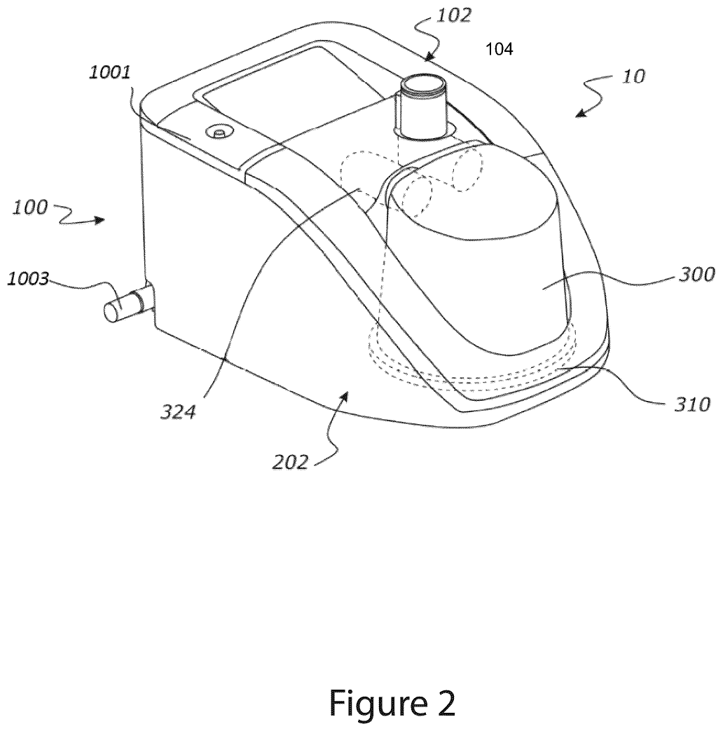

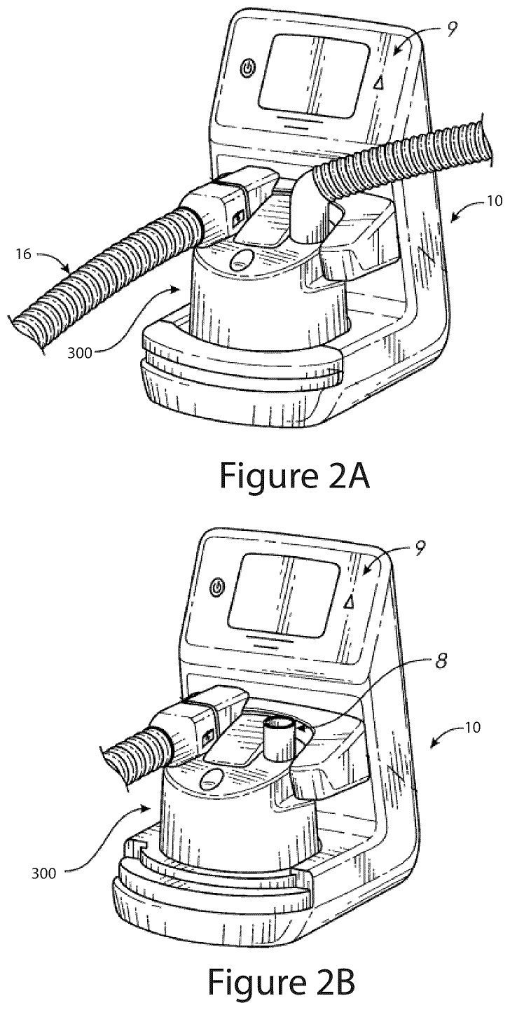

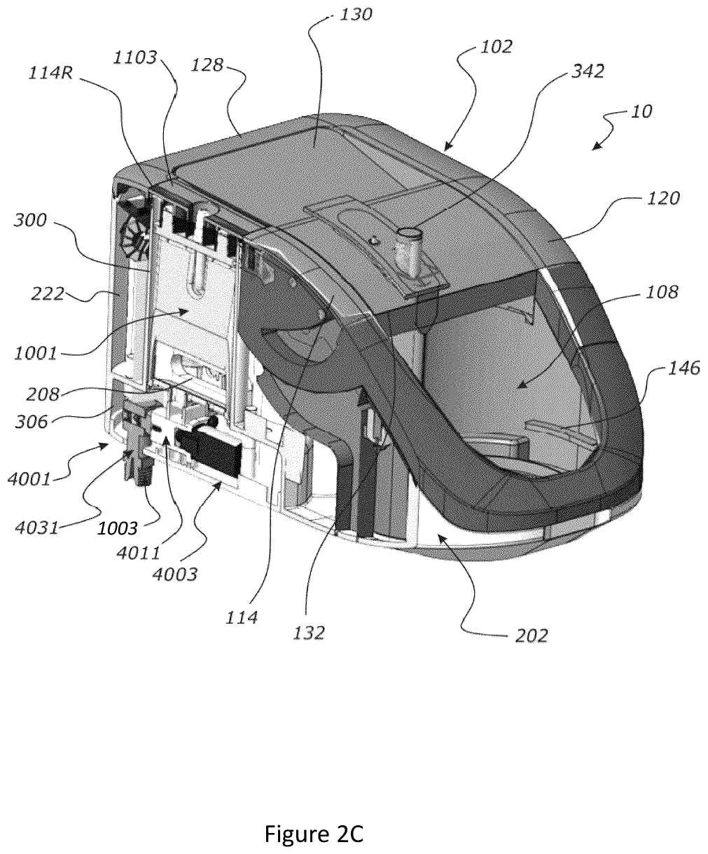

FIG. 2 shows an example breathing assistance or respiratory apparatus 10. The breathing assistance or respiratory apparatus 10 of FIG. 2 can have any of the features of the breathing assistance or respiratory apparatus 10 of FIG. 1. Therefore, features of the breathing assistance or respiratory apparatus 10 of FIG. 1 can be incorporated into the breathing assistance or respiratory apparatus 10 of FIG. 2 and features of the breathing assistance or respiratory apparatus 10 of FIG. 2 can be incorporated into the breathing assistance or respiratory apparatus 10 of FIG. 1.

The breathing assistance or respiratory apparatus 10 of FIG. 2 can include a flow generator and a humidifier. The humidifier can be pneumatically connected to the flow generator. As shown in FIG. 2 the humidifier and flow generator are integrated into a common housing 100. The housing 100 can include a housing upper chassis 102 and a housing lower chassis 202. This configuration provides a compact device that can be easily moved around or carried to provide mobility. Further the flow generator and humidifier being combined in the same housing 100 allows for simpler set up (i.e. a chamber 300 is positioned in the housing 100, such as in a chamber bay 108 (see FIG. 2C)).

The flow generator of the examples of respiratory apparatus disclosed herein can be or comprise a blower module. The blower module may comprise at least one blower configured to generate said flow of gases. The flow generator can include an ambient air inlet port through which ambient room air can be entrained into the blower.

The blower can operate at a motor speed of greater than about 1,000 RPM and less than about 8,000 RPM, greater than about 2,000 RPM and less than about 10,000 RPM, or between any of the foregoing values. The blower can mix the gases entering the blower through the gases inlet (for example, the ambient air inlet port and/or an oxygen inlet port). Using the blower as the mixer can decrease the pressure drop relative to systems with separate mixers, such as static mixers comprising baffles.

The breathing assistance apparatus 10 may also include an oxygen inlet port leading to a valve through which a pressurized gas may enter the flow generator. The valve (which can be the gas flow control 50D of FIG. 1) can control a flow of oxygen into the flow generator. The valve can be any type of valve, including a proportional valve or a binary valve. As shown for example in FIG. 2, the respiratory apparatus 10 can include an oxygen inlet port 1003, which includes a valve through which a pressurized gas (for example, oxygen or any other supplementary gas) may enter the respiratory apparatus 10. The valve can control a flow of oxygen into the respiratory apparatus 10.

The source of oxygen can be an oxygen tank or a hospital oxygen supply. Medical grade oxygen is typically between 95% and 100% purity. Oxygen sources of lower purity can also be used. Examples of valve modules and filters are disclosed in U.S. Publication No. 2019/0255276, titled “Valve Modules and Filter”, which is hereby incorporated by reference in its entirety and should be considered part of the disclosure.

Additional details of the valve will now be described with reference to FIG. 2C. The valve can be in the form of a valve module 4001. The valve module 4001 controls the flow of oxygen and/or other gases entering the gases flow path of the apparatus 10, and enables the apparatus 10 to regulate the proportion of oxygen entrained in the airflow. The valve module may be configured to operate to control the oxygen concentration of the gases provided to the user to at a therapy oxygen concentration. The valve module can be formed as a modular unit for ease of manufacture, assembly, servicing, or replacement. For example, in the event of malfunction, routine maintenance, or future upgrade/improvement. The valve module 4001 can be inserted vertically in an upward direction into a valve module receptacle 306 in the housing lower chassis 202. The valve module can be inserted in other suitable configurations into the housing 100.

The apparatus 10 may comprise a filter module 1001, which may comprise a filter. The filter modules 1001 and valve modules 4001 described herein may provide varying gases flow paths for the apparatus. For example, the valve module 4001 may control the flow of oxygen entering the gases flow path of the apparatus, via the valve module 4001 and filter module 1001. Alternatively, the valve module 4001 may be bypassed by means of direct connection of an alternative oxygen source to the filter module 1001 via an alternative supply inlet. This may be practical in circumstances where a user may wish to manually adjust the oxygen supply (i.e. by a wall-supply rotameter).

The filter modules 1001 and the valve modules 4001 described herein may be used separately in apparatuses for delivering a flow of gases. Alternatively, the filter module and the valve module may be used together as a filter and valve assembly for improved functionality.

A flow control valve 4003 of the valve module 4001 can control a flow of gases through a valve manifold 4011. An end of the valve manifold 4003 can be arranged to receive and connect to a connector 4031. In the form shown, the connector 4031 is a swivel connector. Alternatively, the connector 4031 may be arranged such that oxygen inlet port 1003 can move in a different way, such as a translational movement or pivoting movement for example. The valve 4003 can be arranged to control a flow of gases into part of the apparatus 10. For example, the valve 4003 may be arranged to control a flow of gases to the filter module 1001. Alternatively, the valve 4003 may be arranged to control a flow of gases to another part of the apparatus 10. The valve module 4001 and the filter module 1001 can be positioned upstream of the flow generator and a motor/sensor module. Alternatively, the valve module 4001 and the filter module 1001 can be positioned downstream of the flow generator.

Although the filter module and valve module are described with reference to a breathing assistance apparatus that can deliver heated and humidified gases to a patient or user, the filter module and/or valve module may alternatively be used with an apparatus that does not require a humidifier and therefore does not require the humidification chamber 300. For example, the configuration that isolates the motor and gases flow path from the electrical and electronic components has broad applications in other types of gases delivery apparatuses.

In the configurations disclosed herein, the apparatus 10 receives oxygen by at least one of the following:
via the valve module (for automatic oxygen regulation by the apparatus), or
via the alternative gases inlet provided on the top of the filter (allowing attachment of a manually adjustable oxygen supply-such as a wall supply regulated by a regulator).

The alternative gases inlet may be provided with a therapeutic gas that is not oxygen (for example Heliox)

The apparatus 10 may comprise a manifold. The manifold may be located on the housing. The manifold may provide one or more of: the oxygen inlet, the alternative gases inlet, and/or the air inlet.

The manifold may provide the oxygen, alternative gases, and/or ambient air to the valve module, filter module, and/or the blower.

The manifold may be provided upstream of the blower.

The oxygen inlet or alternative gases supply inlet may be provided on a side of the manifold.

The manifold may allow excess oxygen to overflow to the ambient environment, and/or may allow oxygen to overflow to the ambient environment if the blower is off and oxygen is continually supplied. This prevents accumulation of O₂ in the housing.

The manifold may comprise one or more baffles that help to mix the oxygen and/or the alternative gases and air.

The manifold may also comprise a filter configured to filter the oxygen and/or the alternative gases and/or air from the respective inlets.

The respiratory apparatus 10 can receive supplemental gases including low-pressure supplemental oxygen (“LPO”) and high-pressure supplemental oxygen (“HPO”). A HPO source can be connected via a proportional valve (for example, the gas flow control 50D shown in FIG. 1) that can be electronically controlled by the respiratory apparatus (for example, by its controller) to control the flow rate of oxygen from the HPO source. The LPO source can be fed through a LPO inlet. The flow rate of oxygen from the LPO source may not be controlled by the apparatus. The respiratory apparatus can draw in ambient air for delivery to the patient at high flow rates. The respiratory apparatus can also draw in ambient air and mix it with supplemental gases for delivery to the patient. A user can adjust the FiO₂ value on the user interface, although the quantity actually adjusted is the amount of oxygen in the gases flow provided by the respiratory apparatus, i.e. the fraction of delivered oxygen (FdO₂) may be controlled by the respiratory apparatus. As previously stated, FiO₂ can be close to or equivalent to FdO₂ under certain conditions. Generally, these conditions are desired during operation of the respiratory apparatus. Furthermore, the flow rate and temperature of breathable gases (for example, ambient air and/or an O₂ mixture) delivered by the respiratory apparatus can be controlled similarly (that is, via the user interface).

The breathing assistance or respiratory apparatus 10 may optionally not include a flow generator. In this case the apparatus 10 does not generate a flow of gases, and instead is configured to be connected to an external flow generator and configured to humidify the flow of gases from the external flow generator. For example, the breathing assistance apparatus 10 can be used as a stand-alone humidifier to humidify gases flowing through the humidifier. The flow generator may be wall gas(es) source or a ventilator or other separate flow generator that can be configured to provide one of the therapies described elsewhere in the specification (e.g. NIV, NHF, CPAP, BCPAP, invasive ventilation etc). The humidifier may include a battery coupled to the humidifier to supply power when mains is unavailable (as described in more detail below). The battery may be removably coupled to the apparatus and is rechargeable. The humidifier is pneumatically coupled to a flow generator via a conduit and a separate conduit couples to the humidifier to covey humidified gases from the humidifier to a patient. The breathing assistance apparatus 10 may for example be the apparatus as described in International Application No. WO 2015/093989, which is herein incorporated by reference in its entirety and should be considered part of the disclosure.

An example of a breathing assistance apparatus 10 as a stand-alone humidifier (i.e. without a flow generator) is shown in FIGS. 2A and 2B. The breathing assistance apparatus 10 can include any of the features of the breathing assistance apparatus 10 of FIGS. 1 and 2, with the differences described below with reference to FIGS. 2A and 2B. The humidifier as shown in figures s! and 2B can also be used to communicate with a peripheral device and perform the simulation as described. The apparatus includes a connector that pneumatically connects a conduit 16 (as described in more detail above) to an outlet (as a gases outlet of the apparatus) of a humidification chamber 300. The conduit 16 may be an inspiratory limb of a patient circuit, i.e., configured to deliver humidified gases to a user, such as via a patient interface (not shown). The conduit 16 may have a conduit heater (as for example described elsewhere in the specification).

An inlet 324 of the humidification chamber 300 is configured to be fluidly connected to a flow generator positioned remote from the apparatus 10 (for example by the conduit shown connected to inlet 324 in FIG. 2A).

As shown in FIGS. 2A and 2B the apparatus 10 further includes a panel 9 which may be used to mount a user display and/or controls 54. For example, various dials, switches, and other input means may be used to control operation of the respiratory apparatus. Additionally, or alternatively, a touch screen display may be used. The user display may display parameters of the system, warnings in the event of any errors or malfunctions, or prompts where user action is required, etc. Where a touch screen display is used, the same display may be used to present information to a user and receive inputs from a user, at least in part (as for example described elsewhere in the specification). The humidifier apparatus shown in FIGS. 2A and 2B may comprise multiple sensors. For example, the humidifier may comprise a flow sensor, one or more temperature sensors, one or more pressure sensors and one or more humidity sensors. In one example configuration the humidifier comprises at least a temperature sensor positioned within or adjacent the inlet and a temperature sensor within or adjacent the outlet of the chamber 300. Optionally the humidifier may comprise a flow sensor within or adjacent the outlet of the chamber 300. Additionally, a further flow sensor may be located within or adjacent the inlet 324. Optionally the humidifier may include a single flow sensor located in the inlet or outlet. The humidifier may additionally comprise one or more humidity sensors, that may be arranged within or adjacent either of the inlet or outlet, or in the inlet and outlet. Further there may be additional ambient temperature sensors. The humidifier may also comprise additional sensors associated with the heater e.g. temperature sensors associated with the heater.

In the examples of the breathing assistance or respiratory apparatus 10 disclosed herein, the humidifier may comprise a humidification chamber 300. The liquid in the humidification chamber may be water or another liquid, and/or may comprise a mixture of one or more liquids (for example a mixture of water and a medicament). The humidification chamber 300 may be configured to be removed from the humidifier (for example for replacement, cleaning and/or refilling). Alternatively, the humidification chamber may be non-removable from the humidifier.

The humidification chamber 300 may comprise an autofill mechanism that includes at least a valve and a float coupled to the valve. The humidification chamber 300 in use can be coupled to water reservoir of water bag to auto fill. Alternatively, the humidification chamber 300 may be manually refilled.

The humidifier may comprise a humidifier heater 310 for example as a heater plate (see FIG. 2). The heater 310 of the humidifier 12 may be an electrically conductive heating element. The humidifier heater 310 provides heat to the humidification chamber 300. The humidifier can humidify the gases flow and/or heat the gases flow to an appropriate level. As will be described in greater details below, the apparatus 10 can include a controller that can control the humidifier (for example by controlling at least a humidifier heater).

With continued reference to FIG. 2, a breathing conduit 16 is coupled at one end to a gases outlet 104 in the housing 100 of the breathing assistance apparatus 10. The breathing conduit 16 is coupled at another end to a patient interface such as a non-sealed nasal cannula with a manifold and nasal prongs. Additionally, or alternatively, the breathing conduit 16 can be coupled to a face mask, a nasal mask, a nasal pillows mask, an endotracheal tube, a tracheostomy interface, and/or the like. The gases flow that is generated by the breathing assistance apparatus 10 may be humidified and delivered to the patient via the breathing conduit 16 and the patient interface.

A breathable conduit may be provided between the breathing conduit 16 and the patient interface. Alternatively, the breathing conduit 16 may include breathable material(s).

A different conduit type may be connected to the gases outlet 102, for example a disinfection conduit in a disinfection mode. To use the respiratory apparatus 10 disclosed herein on multiple patients, at least certain components of the apparatus 10 must have a high level disinfection process (by operating the apparatus 10 in the disinfection mode) carried out between different patients using it. The disinfection conduit can be heated gases tube. The disinfection conduit can be used with the apparatus 10 instead of the breathing conduit 16 to allow for disinfection of certain components during the disinfection mode.

The breathing conduit 16 can have a heater to heat the gases flow passing through to the patient. The heater can be under the control of the controller. In at least one configuration, the heater is a heater wire. The breathing conduit 16 and/or patient interface can be considered part of the breathing assistance system. The breathing assistance system 10 may comprise the breathing assistance apparatus 10, breathing conduit 16, and patient interface.

The heater of the breathing conduit may be located:
a) in a lumen of the breathing conduit 16,
b) within the wall of the breathing conduit 16,
c) embedded in a wall of the breathing conduit 16,
d) embedded in a bead which forms the breathing conduit 16, (The bead can be configured to provide structural support to the conduit 16.)
e) located on an external surface of the breathing conduit 16, or
f) any combination of a)-e).

The heater may extend linearly along the conduit 16, or be helically wrapped around the conduit or be helically wrapped within the conduit.

The heater of the breathing conduit 16 may be an electrically conductive heating element (for example a heater wire).

As described above, the controller can control the flow generator to generate a gases flow at the desired flow rate (for example a therapy flow rate). The controller can also control a supplemental oxygen inlet to allow for delivery of supplemental oxygen.

The controller can also control a humidifier heater in the humidifier and/or the heater in the breathing conduit 16 to heat the gas(es) to a desired temperature for a desired level of therapy and/or level of comfort for the patient.

The controller may comprise one or more processors. The processors may be configured with computer-readable instructions. The controller may be a microprocessor or an ASIC, FPGA or a combination of ICs or microprocessors or other suitable components and/or architectures. The controller may comprise at least one memory device. The memory device may be configured to store said computer-readable instructions. The memory device may be non-transitory computer readable medium. The instructions, when executed by the one or more processors cause the respiratory therapy apparatus to affect the steps and processes described herein. It will be appreciated that when the specification describes the apparatus 10 undertaking an action, it may be that the controller is controlling one or more components of the apparatus 10 to undertake the action. It will be appreciated the methods described herein can be executed by the controller (or another processor).

The controller can be configured or programmed to control the operation of the breathing assistance apparatus 10. For example, the controller can control components of the breathing assistance apparatus 10, including but not limited to: operating the flow generator to create a flow of gas(es) (gases flow) for delivery to a patient, operating the humidifier (if present) to humidify and/or heat the generated gases flow, controlling a flow of oxygen into the flow generator blower, receiving user input from the user interface 54 for reconfiguration and/or user-defined operation of the breathing assistance apparatus 10, and outputting information (for example on the display) to the user.

The controller may comprise one or more sub controllers. The sub controllers may each be configured to control one or more components of the apparatus (for example a flow generator sub controller, and/or a humidifier sub controller and/or a humidifier or conduit heater sub controller). The controller may include a master controller configured to communicate with, and pass commands to the sub controllers.

The methods described herein may be embodied as software or a software module as part of control software (for example computer-readable instructions) that are stored in the controller (or associated memory) and executed by the controller (and/or an associated processor).

The controller can be provided with or can determine a suitable target temperature of the gases flow. The controller may control the humidifier heater of the humidifier and/or the heater of the breathing conduit based on one or more suitable target temperature(s) of the gases flow.

The heater of the breathing conduit 16 may be controlled by the controller to reach a desired temperature. The desired temperature may be, or be based on, one or more temperature set points, and/or one or more humidity set points (for example a therapy humidity). The controller may control the heater of the breathing conduit 16 based on a desired temperature of the gases at the patient interface and/or a desired temperature at the end of the breathing conduit 16. The desired temperatures may be at end of the breathing conduit 16, at the patient interface, at the gases outlet, a humidification chamber outlet, at any sensor of the apparatus, and/or any combination thereof. The one or more temperature set points may comprise one or more of:
a desired dew point (for example a temperature indicative of a desired humidity),
a predetermined dew point,
a predetermined temperature, or
a desired temperature.

The humidifier heater of the humidifier may be controlled by the controller to reach a desired temperature. The desired temperature may be, or be based on, one or more temperature set points, and/or one or more humidity set points. The desired temperature may be a therapy parameter.

The controller may control the heater of the breathing conduit 16 and/or the humidifier heater of the humidifier to the desired temperature by closed loop control based on the output of one or more sensors.

The one or more temperature set points may relate to one or more therapy parameters of the apparatus for therapy (for example a dew point or temperature of the gases), or be provided in the memory of the apparatus (for example a predetermined temperature).

The controller 13 may be configured to control the heater of the humidifier and/or the heater of the conduit according to a first control scheme and a second control scheme. The controller 13 may transmit commands to humidifier heater sub controller and/or the conduit heater sub controller. The first control scheme is a first power control scheme. The second control scheme is a second power control scheme. When the apparatus is powered by the battery 125 the controller is configured to control the heater of humidifier and/or the heater of the conduit according to a first control scheme.

The first control scheme may comprise providing the heater of the humidifier with a low frequency pulse-width modulation signal. The second control scheme may comprise providing the heater of the humidifier with a high frequency pulse-width modulation signal. The frequency of the high frequency pulse-width modulation signal is a greater than the frequency of the low frequency pulse-width modulation signal.

In some configurations, when the apparatus is powered by an external supply, the controller may be configured to control the heater of the humidifier and/or the heater of the conduit according to a second control scheme.

The second control scheme may comprise controlling the heater of the humidifier by digital control.

Additionally, or alternatively, the second control scheme may comprise controlling the heater of the conduit by digital control.

The first control scheme may comprise providing the heater of the humidifier with a high frequency pulse-width modulation signal.

The second control scheme may comprise providing the heater of the humidifier with a low frequency pulse-width modulation signal.

The frequency of the high frequency pulse-width modulation signal is a greater than the frequency of the low frequency pulse-width modulation signal.

Details of the first and second control schemes are described in International Application No. PCT/IB2022/054939, filed 26 May 2022 and titled “CONTROL OF COMPONENTS OF A BREATHING ASSISTANCE APPARATUS,” the disclosure of which is hereby incorporated herein by reference in its entirety and should be considered part of the disclosure.

As described above, the apparatus may provide different therapy modes including but not limited to any combination of: Nasal High Flow (NHF) therapy, Continuous Positive Airway Pressure (CPAP) therapy, Non-Invasive Ventilation (NIV) (e.g., BiPAP) and Bubble Continuous Positive Airway Pressure (BCPAP) therapy. The apparatus may comprise one or more control modes associated with each therapy type. The control modes may be manually selected by the user or automatically selected depending on the components connected to the apparatus (for example dependent on the type of tube and/or patient interface connected to the apparatus). Each control mode may have an associated control scheme for controlling components of the apparatus (for example the flow generator, humidifier heater 310 or conduit heater 16a).

The one or more therapy parameters for NHF therapy may comprise any combination of:
a therapy flow rate of the gases provided to the user,
a therapy humidity level (for example a relative or absolute humidity, or a dew point),
a therapy oxygen concentration provided to the user (for example, FiO₂ or FdO₂),
a therapy concentration of an auxiliary gas provided to the user, or
a therapy temperature of the gases provided to the user.

The one or more therapy parameters for BCPAP therapy may comprise any combination of:
a therapy flow rate of the gases provided to the user,
a therapy humidity level (for example a relative or absolute humidity, or a dew point),
a therapy oxygen concentration provided to the user,
a therapy concentration of an auxiliary gas provided to the user, or
a therapy temperature of the gases provided to the user.

The one or more therapy parameters for CPAP therapy may comprise any combination of:
a therapy humidity level (for example a relative or absolute humidity, or a dew point)
a therapy oxygen concentration provided to the user,
a therapy temperature of the gases provided to the user.
a therapy concentration of an auxiliary gas provided to the user,
a therapy level of pressure support (for example a CPAP pressure) provided to the user
a therapy PEEP provided to the user.

The one or more therapy parameters for Bi-level PAP therapy, i.e., NIV therapy may comprise any combination of:
a therapy humidity level (for example a relative or absolute humidity, or a dew point)
a therapy oxygen concentration provided to the user,
a therapy temperature of the gases provided to the user.
a therapy concentration of an auxiliary gas provided to the user,
a therapy IPAP/EPAP (inspiratory positive airway pressure/expiratory positive airway pressure) provided to the user.

The therapy temperature may comprise a therapy temperature at the chamber outlet and/or a therapy temperature at the end of the breathing conduit.

The therapy humidity may be at the chamber outlet or at the end of the breathing conduit.

The therapy humidity level may be a dew point of about 27 degrees Celsius to about 40 degrees Celsius, or about 29 degrees Celsius to about 39 degrees Celsius, or about 31 degrees Celsius to about 38 degrees Celsius, or about 37 degrees Celsius, or an absolute humidity of above about 38 mg/L to about to 44 mg/L.

Providing humidity to a user can increase patient comfort and compliance with therapy. The provision of humidity also provides additional benefits of improving mucus transport which is useful in patient with obstructive pulmonary diseases, improving comfort and therefore compliance/acceptance of these therapies.

The user may enter one or more therapy parameters via a user interface. The breathing assistance apparatus may comprise at least one display module, configured to display an alarm output. The display module may comprise at least one display (for example a liquid crystal display (LCD), or a light emitting diode (LED) display, although it will be appreciated any display technology may be used). The display module may be configured to receive inputs to the system (for example as a touch screen) and therefore be at least part, or display part of the user interface (for example, the user interface 54).

The display module may be configured to be an input/output (I/O) module (for example, the I/O module 54 of FIG. 1). For example, the display module may be configured to receive inputs from a user and provide outputs to a user (for example as part of, or to display part of the user interface 14).

The display module may communicate with the controller. The display module may provide information to the controller (for example set points). The display module may receive information from the controller (for example alarms, sensor outputs, and/or other calculated variables).

The breathing assistance apparatus may comprise at least one audible module configured to emit an audible alarm. The at least one audible module may comprise a speaker.

The apparatus may be powered by a mains voltage power supply (for example a wired connection with an electrical grid or for example a portable electrical generator, distributed generation source, and/or a non-portable electrical generator such as hospital back-up generators).

Mains power supply may be any power supply configured to be connected to the apparatus via the electrical socket.

Mains power supply may include to any power supply which does not have a capacity constraint (for example as with a battery).

The apparatus may be powered by a non-peak power limited supply or a peak power limited supply (for example a battery). Peak power limited in this context refers to the peak power required by the apparatus to operate with full capability.

The apparatus may be powered by an integrated power supply or an external power supply.

Sensors such as flow, temperature, humidity, and/or pressure sensors (for example, the sensors 53A-C shown in FIG. 1), can be placed in various locations in the respiratory apparatus 10. Additional sensors (for example, sensors 53D and 14 shown in FIG. 1) may be placed in various locations on the breathing conduit 16 and/or patient interface (for example, there may be a temperature sensor at or near the end of the inspiratory tube).

The respiratory apparatus 10 may have a communications module to enable the controller of the respiratory apparatus 10 to receive signals from the sensors and/or to control the various components of the respiratory apparatus 10, including but not limited to the flow generator, humidifier, delivery conduit heater, humidifier heater, or accessories or peripherals associated with the respiratory apparatus 10. Additionally, or alternatively, the communications module may deliver data to a remote server or enable remote control of the respiratory apparatus 10 or respiratory system.

The communications module may comprise a transmitter, receiver and/or transceiver.

The communications module may act as a network interface (for example as a modem).

The communications module may use one or more communication protocols known in the art, for example Wi-Fi, Bluetooth, Zigbee, cellular (3G, 4G, or 5G etc).

The communications module may allow for communication between the apparatus and a mobile device (for example a phone or a tablet via Bluetooth or Wi-Fi)

The communications module may comprise a number of separate transmitters, receivers and/or transceiver for each, or for a group of communication protocol(s).

The communications module may be configured to transmit data and receive data from one or more devices (for example a server) as described in more detail below.

The communications module may transmit one or more leak or blockage events, or alarms (as described in more detail below) to one or more servers and/or devices (for example a computer, phone or tablet). Additional information (for example the time, duration, or severity) associated with the event or alarm may be additionally transmitted to the server and/or device by the communications module.

As described above, the breathing assistance apparatus 10 can measure and control the oxygen content of the gases being delivered to the patient. The control can be via a closed-loop oxygen control system. Patients suffering from various health conditions and diseases can benefit from oxygen therapy. For example, patients suffering from chronic obstructive pulmonary disease (COPD), pneumonia, asthma, bronchopulmonary dysplasia, heart failure, cystic fibrosis, sleep apnea, lung disease, trauma to the respiratory system, acute respiratory distress, receiving pre- and post-operative oxygen delivery, and other conditions or diseases can benefit from oxygen therapy. A common way of treating such problems is by supplying the patients with supplemental oxygen to prevent their blood oxygen saturation (SpO₂) from dropping too low (e.g., below about 90%). However, supplying the patient with too much oxygen can over oxygenate their blood, and is also considered dangerous. Generally, the patient's SpO₂ is kept in a range from about 80% to about 99%, and preferably about 92% to about 96%, although these ranges may differ due to patient conditions. Due to various factors such as respiratory rate, lung tidal volume, heart rate, activity levels, height, weight, age, gender, and other factors, there is no one prescribed level of supplemental oxygen that can consistently achieve an SpO₂ response in the targeted range for each patient. Individual patients will regularly need their fraction of oxygen delivered to the patient (FdO₂) monitored and adjusted to ensure they are receiving the correct FdO₂ to achieve the targeted SpO₂. Achieving a correct and consistent SpO₂ is an important factor in treating patients with various health conditions or diseases. Additionally, patients suffering from these health problems may find benefit from a system that automatically controls oxygen saturation. The present disclosure is applicable to a wide range of patients that require fast and accurate oxygen saturation control.

The fraction of oxygen delivered to a patient (FdO₂) may be controlled manually. A clinician can manually adjust an oxygen supply valve to change the flow rate or fraction of oxygen being delivered to the patient. The clinician can determine SpO₂ levels of the patient using a patient monitor, such as a pulse oximeter. The clinician can continue to manually adjust the amount of oxygen being delivered to the patient until the SpO₂ level of the patient reaches a determined level.

One problem with current methods is that when the clinician is trying to achieve a specific SpO₂ level they would need to alter the FdO₂, wait for the SpO₂ reading to settle, and then apply further changes to the FdO₂ until the SpO₂ is at the required level. The repetitive process of altering the FdO₂ and waiting for the SpO₂ to settle can be a very time-consuming process, particularly if multiple patients are requiring the same treatment.

Another problem is related to the accuracy of the SpO₂ that can be achieved. Accuracy of the SpO₂ control can be dependent on how fine the increments are for the displayed SpO₂ and selectable FdO₂. The accuracy may be hampered by the increased amount of time required to get increasingly accurate values, as a clinician may get close to the ideal SpO₂ and decide not to alter the FdO₂ any further.

Another problem is that other factors may cause the patient's SpO₂ levels to change over time without any change in FdO₂. Patients would need to be regularly checked on and have their FdO₂ adjusted in order to maintain their SpO₂ at the correct value. This process can be quite time consuming for the clinician. Additionally, if the time between adjustments is too long, the patient can be at risk of their SpO₂ drifting too far from the targeted level.

While some systems exist that attempt automatic SpO₂ control similar to the current methods described above, many of them are plagued by further problems stemming from difficulties in measuring patient oxygen saturation. Pulse oximeters and similar devices generate a signal that lags far behind the corresponding change in oxygen fraction delivered. Additionally, oxygen saturation readings can become inaccurate due to various factors.

The present disclosure provides for closed loop control of a respiratory apparatus that allows a patient or clinician to set a target SpO₂ instead of a target FdO₂. The respiratory apparatus can automatically alter the FdO₂ of the respiratory apparatus to achieve the targeted SpO₂ based on values of target SpO₂, current SpO₂, and current FdO₂. Automatically controlling the FdO₂ can help to quickly and accurately adjust the FdO₂ until a target SpO₂ is achieved. In some configurations, the system can generate a patient specific model for each patient at the initiation of a therapy session. The respiratory apparatus can have greater precision in achieving the targeted SpO₂ by adjusting the FdO₂, as needed, to stay within the targeted SpO₂ range, without being constantly monitored by a clinician.

The present disclosure provides for a respiratory apparatus that can implement one or more closed loop control systems. Features of the closed loop control system may be combined with features of one or more configurations of the respiratory apparatus or system disclosed herein.

The respiratory apparatus may operate in automatic mode or manual mode. In automatic mode, the controller can automatically control the FdO₂ based on a target FdO₂ determined based on the target SpO₂ and/or measured SpO₂. A valve at the oxygen inlet (such as the valve 4003 of FIG. 2C) may be connected to the controller that can control the oxygen concentration in gases flow based on a target FdO₂. The controller can execute a control algorithm that can measure FdO₂ output by the respiratory apparatus. The FdO₂ measurement may be taken periodically at a defined frequency, such as a maximum sample rate of the gases concentration sensors or at a lower frequency, or the measurement may be taken aperiodically. The controller can continue to adjust the valve at the oxygen inlet until the measured FdO₂ arrives at the target FdO₂. The measured FdO₂ may be determined by a gases composition sensor.

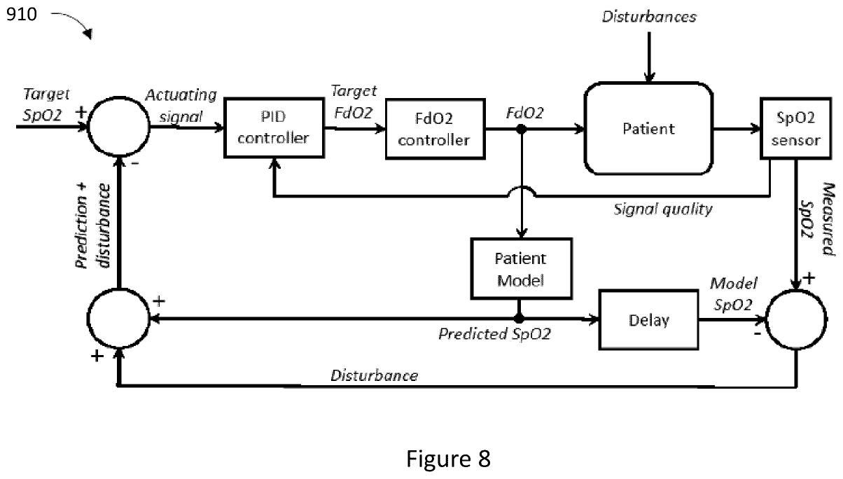

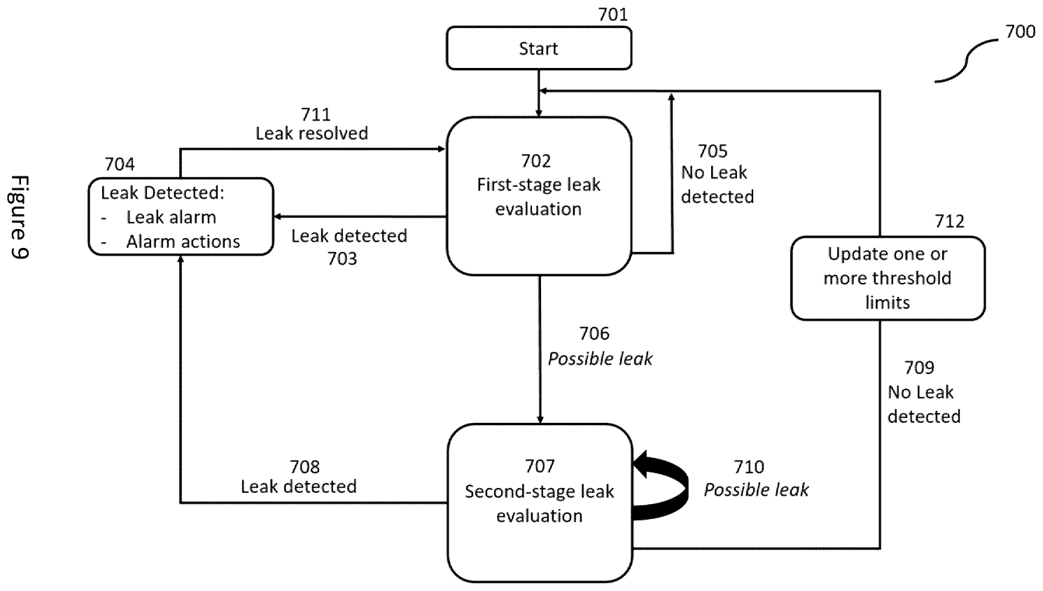

In the automatic mode, the controller can implement a PID controller to control the FdO₂ as disclosed herein. FIG. 8 illustrates a schematic diagram 910 of a Smith predictor being utilized with a PID controller of the respiratory apparatus disclosed herein. Initially, the PID controller receives an input of the difference between the predicted patient SpO₂ and target SpO₂, and outputs a target FdO₂ to the FdO₂ controller to bring the patient SpO₂ closer to the target SpO₂. The FdO₂ controller outputs instructions to control the valve of the flow therapy apparatus based on the target FdO₂ output by the PID controller.

In manual mode, the controller can receive a target FdO₂ from a user (for example, a clinician or patient), such as via a user interface (for example, the user interface 54 of FIGS. 1-2B). The controller can automatically control the FdO₂ based on the received target FdO₂. The controller can control the oxygen concentration in gases flow by controlling the oxygen inlet valve based on a target FdO₂. The controller can execute a control algorithm that can use a measured FdO₂ output by the respiratory apparatus (for example, by a gases composition sensor of the respiratory apparatus) as an input to the controller. The FdO₂ measurement may be taken periodically at a defined frequency, such as a maximum sample rate of the gases concentration sensors or at a lower frequency, or the measurement may be taken aperiodically. The controller can continue to adjust the valve at the oxygen inlet to drive the measured FdO₂ towards the target FdO₂. The measured FdO₂ may be determined by a gases composition sensor.

The respiratory apparatus may be configured to change from automatic mode to manual mode when the SpO₂ of the patient is not within an acceptable patient range. In some instances, the respiratory apparatus reverts to manual mode when the SpO₂ of the patient is outside of the patient limits (above or below) or if the patient's SpO₂ did not move within the limits within a defined period of time after the start of the therapy session. The respiratory apparatus may revert to manual mode when the signal quality of the patient sensor is below a threshold level for a defined period of time. In some configurations, the respiratory apparatus may trigger an alarm when it switches from automatic mode to manual mode. In some configurations, the respiratory apparatus may trigger an alarm when the signal quality of the patient sensor is below a threshold level for a defined period of time. The respiratory apparatus may continue to function in automatic mode after the alarm is triggered. The respiratory apparatus may provide the user, through a graphical user interface, with an option to disable the alarm or to exit automatic mode.

In the automatic mode, the controller may utilize two control loops. The first control loop can determine a target FdO₂ based on the target SpO₂. The second control loop can use the target FdO₂ output by the first control loop and measured FdO₂ to output an oxygen inlet valve control signal. In the manual mode the controller may only use the second the control loop, and the second control loop can receive a target FdO₂ output from user input or a default value.

During a high flow therapy session, the oxygen concentration measured in the apparatus, fraction of delivered oxygen (FdO₂), can be substantially the same as the oxygen concentration the user is breathing, fraction of inspired oxygen (FiO₂), when the flow rate of gases delivered meets or exceeds the peak inspiratory demand of the patient. This means that the volume of gases delivered by the apparatus to the patient during inspiration meets, or is in excess of, the volume of gases inspired by the patient during inspiration. High flow therapy helps to prevent entrainment of ambient air when the patient breathes in, as well as flushing the patient's airways of expired gas. So long as the flow rate of delivered gases meets or exceeds peak inspiratory demand of the patient, entrainment of ambient air is prevented and the gas delivered by the respiratory apparatus, FdO₂, is substantially the same as the gas the patient breathes in, FiO₂.

The breathing assistance apparatus may further comprise a gases composition sensor. The gas composition sensor may be the sensor described below (for example the ultrasonic transducer configuration). The breathing assistance apparatus 10 comprises a flow sensor. For example, the flow sensor can be the examples of flow rate sensors described below. The flow sensor may be configured to measure a flow rate of the flow of breathable gases to a patient.

The respiratory apparatus 10 may include a motor and/or a sensor sub-assembly (for example containing one or more sensors) in the housing.

The motor and/or sensor sub-assembly may be located recess on in the underside of the housing. The recess may alternatively be in the rear, side, front, or top of the housing. The air and/or oxygen inlets may also be positioned differently as required.

As another example, rather than the humidification chamber and chamber bay being configured so that the humidification chamber is inserted into and removed from the chamber bay from a front of the housing, the configuration could be such that the humidification chamber is inserted into and removed from the chamber bay from a side, rear, or top of the housing.

As another example, while the filter modules are described as being inserted into the housing from above and the valve modules inserted into the housing from below, either or both of those components could be inserted into any suitable part of the housing, such as an upper part, lower part, side part, front part, or rear part.

Oxygen may be measured by one or more gases composition sensors (such as an ultrasonic transducer system) located downstream of mixing of the oxygen and ambient air. The measurement can be taken within the respiratory apparatus 10, the patient breathing conduit 16, the patient interface, or at any other suitable location. The gases composition sensors can be located on a sensing circuit board. The motor and/or sensor subassembly of the respiratory apparatus 10 described above can house the sensing circuit board, which can be a printed sensing circuit board (PCB).

The sensing circuit board can comprise ultrasonic transducers, transceivers, or sensors of the sensing circuit board to measure gases properties of the gases flow, such as gases composition or concentration of one or more gases within the gases stream. Any suitable transducer, transceiver, or sensor may be mounted to the sensing circuit board. The sensing circuit board can include an ultrasonic transducer system (also referred to as an ultrasonic sensor system) that employs ultrasonic or acoustic waves for determining gas concentrations. Various sensor configurations are described below.

The ultrasonic transducer system may determine the relative gas concentrations of two or more gases in the gases flow. The ultrasonic transducer system may be configured to measure the oxygen fraction in the bulk gases stream flow, which includes atmospheric air augmented with supplemental oxygen, which is essentially a binary gas mixture of nitrogen (N₂) and oxygen (O₂). The ultrasonic transducer system may be configured to measure the gases concentrations of other supplemental gases that have blended with atmospheric air in the gases stream, including nitrogen (N₂) and carbon dioxide (CO₂). The ultrasonic transducers can determine the concentration of gases in the gases flow at a relatively high frequency. For example, the ultrasonic transducers can output a measured FdO₂ value at a maximum sample rate of the sensors or at a lower frequency than the maximum sample rate, such as between about 1 Hz and 200 Hz, about 1 Hz and 100 Hz, about 1 Hz and 50 Hz, and about 1 Hz and 25 Hz.

In some configurations, sensing circuit board can include two ultrasonic transducers that are provided on opposite sides of the sensing circuit board. Various alternative configurations of the ultrasonic transducers can be used for sensing the characteristics of the gases stream by the transmission and reception of ultrasonic beams or pulses.

The oxygen concentration measured in the apparatus may be equivalent to the fraction of delivered oxygen (FdO₂) and may be substantially the same as the oxygen concentration the patient is breathing, the fraction of inspired oxygen (FiO₂) under certain conditions. As described above, generally, FdO₂ and FiO₂ can be seen as equivalent when the flow rate of gases provided by the apparatus meets or exceeds the patient's inspiratory demand—i.e., the amount of ambient air entrainment is minimal.

Oxygen concentration may also be measured by using flow rate sensors on at least two of: the ambient air inlet conduit, the oxygen inlet conduit, and the patient breathing conduit to determine the flow rate of at least two gases. By determining the flow rate of both inlet gases or one inlet gases and one total flow rate, along with the assumed or measured oxygen concentrations of the inlet gases (about 20.9% for ambient air, about 100% for oxygen), the oxygen concentration of the final gases composition can be calculated. Alternatively, flow rate sensors can be placed at all three of the ambient air inlet conduit, the oxygen inlet conduit, and the breathing conduit to allow for redundancy and testing that each sensor is working correctly by checking for consistency of readings. Other methods of measuring the oxygen concentration delivered by the breathing assistance or respiratory apparatus 10 can also be used.

The breathing assistance apparatus 10 of the present disclosure can include a patient sensor (for example, the sensor 14 shown in FIG. 1), such as a pulse oximeter or a patient monitoring system, to measure one or more physiological parameters of the patient, such as a patient's blood oxygen concentration (for example, peripheral blood oxygen saturation (SpO₂), heart rate, respiratory rate, perfusion index, and provide a measure of the signal quality thereof. The sensor can communicate with the controller through a wired connection or by communication through a wireless transmitter on the sensor. The sensor may be a disposable adhesive sensor designed to be connected to a patient's finger. The sensor may be a non-disposable sensor (i.e., a re-useable sensor). Sensors that are designed for different age groups and to be connected to different locations on the patient are available and can be used with the breathing assistance apparatus 10. The pulse oximeter can be attached to the patient, typically at their finger, although other places such as an earlobe are also an option. The pulse oximeter can be connected to a controller (for example, controller 19 as shown in FIG. 1) in the respiratory therapy apparatus 10 and can (for example, constantly) provide signals indicative of the patient's blood oxygen saturation. The patient sensor can be a hot-swappable device, which can be attached or interchanged during operation of the breathing assistance apparatus 10. For example, the patient sensor may connect to the breathing assistance apparatus 10 using a USB interface or using wireless communication protocols (such as Bluetooth®).

When the patient sensor is disconnected during operation, the breathing assistance apparatus 10 may continue to operate in its previous state of operation for a defined time period. After the defined time period, the breathing assistance apparatus 10 may trigger an alarm, transition from the automatic mode (as described above) to the manual mode (as described above), and/or exit control mode (e.g., the automatic mode or the manual mode) entirely. The patient sensor may be a bedside monitoring system or other patient monitoring system that communicates with the breathing assistance apparatus 10 through a physical or wireless interface.

As described above, the breathing assistance apparatus 10 may comprise or be in the form of a high respiratory apparatus. High flow therapy as discussed herein is intended to be given its typical ordinary meaning as understood by a person of skill in the art and generally refers to a breathing assistance apparatus delivering a targeted flow of humidified respiratory gases via an intentionally unsealed (also referred to as non-sealing) patient interface. In some instances, high flow therapy is delivered at flow rates generally intended to meet or exceed inspiratory demand of a patient. Typical patient interfaces include, but are not limited to, a nasal or tracheal patient interface. Typical flow rates for adults often range from, but are not limited to, about fifteen litres per minute to about sixty litres per minute or greater. Typical flow rates for paediatric patients (such as neonates, infants and children) often range from, but are not limited to, about one litre per minute per kilogram of patient weight to about three litres per minute per kilogram of patient weight or greater. High flow therapy can also optionally include gases mixture compositions including supplemental oxygen and/or administration of therapeutic medicaments. High flow therapy is often referred to as nasal high flow (NHF), humidified high flow nasal cannula (HHFNC), high flow nasal oxygen (HFNO), high flow therapy (HFT), or tracheal high flow (THF), among other common names.

For example, in some configurations, for an adult patient ‘high flow therapy’ may refer to the delivery of gases to a patient at a flow rate of greater than or equal to about 10 litres per minute (10 LPM), such as between about 10 LPM and about 100 LPM, or between about 15 LPM and about 95 LPM, or between about 20 LPM and about 90 LPM, or between about 25 LPM and about 85 LPM, or between about 30 LPM and about 80 LPM, or between about 35 LPM and about 75 LPM, or between about 40 LPM and about 70 LPM, or between about 45 LPM and about 65 LPM, or between about 50 LPM and about 60 LPM. In some configurations, for a neonatal, infant, or child patient, ‘high flow therapy’ may refer to the delivery of gases to a patient at a flow rate of greater than 1 LPM, such as between about 1 LPM and about 25 LPM, or between about 2 LPM and about 25 LPM, or between about 2 LPM and about 5 LPM, or between about 5 LPM and about 25 LPM, or between about 5 LPM and about 10 LPM, or between about 10 LPM and about 25 LPM, or between about 10 LPM and about 20 LPM, or between about 10 LPM and 15 LPM, or between about 20 LPM and 25 LPM. A respiratory apparatus that can provide high flow therapy to an adult patient, a neonatal, infant, or child patient, may, in some configurations, deliver gases to the patient at a flow rate of between about 1 LPM and about 100 LPM, or at a flow rate in any of the sub-ranges outlined above. Gases delivered may comprise a percentage of oxygen. In some configurations, the percentage of oxygen in the gases delivered may be between about 20% and about 100%, or between about 30% and about 100%, or between about 40% and about 100%, or between about 50% and about 100%, or between about 60% and about 100%, or between about 70% and about 100%, or between about 80% and about 100%, or between about 90% and about 100%, or about 100%, or 100%.

High flow therapy may be effective in meeting or exceeding the patient's inspiratory flow, increasing oxygenation of the patient, and/or reducing the work of breathing.

High flow therapy may be administered to the nares of a patient and/or orally, or via a tracheostomy interface. High flow therapy can be delivered with a non-sealing patient interface such as, for example, a nasal cannula.

High flow therapy may generate a flushing effect in the nasopharynx such that the anatomical dead space of the upper airways is flushed by the high incoming gases flow. This can create a reservoir of fresh gases available for each and every breath, while reducing re-breathing of nitrogen and carbon dioxide. Meeting inspiratory demand and flushing the airways is additionally important when trying to control the patient's SpO₂. As described above, meeting the inspiratory demand may be desirable at least because FdO₂ (measuring what is delivered by the respiratory apparatus) is approximately equal to FiO₂, which measures what is actually inspirated by the patient). High flow therapy may slow down respiratory rate of the patient.

High flow therapy may be used to treat patients with obstructive pulmonary conditions e.g., COPD, bronchiectasis, dyspnea, cystic fibrosis, emphysema and/or patients with respiratory distress or hypercapnic patients.

The term “non-sealing patient interface” (i.e., unsealed patient interface) as used herein can refer to an interface providing a pneumatic link between an airway of a patient and a gases flow source (such as from flow generator) that does not completely occlude the airway of the patient. A non-sealed pneumatic link can comprise an occlusion of less than about 95% of the airway of the patient. The non-sealed pneumatic link can comprise an occlusion of less than about 90% of the airway of the patient. The non-sealed pneumatic link can comprise an occlusion of between about 40% and about 80% of the airway of the patient. The airway can include one or both nares of the patient and/or their mouth. For a nasal cannula the airway is through the nares.

The “non-sealing patient interface” may comprise a tracheal interface.

A sealed interface may be used when the apparatus is providing CPAP, NIV (e.g., Bilevel PAP) or BCPAP therapy.

Some examples of respiratory apparatuses are disclosed in International Application Nos. WO 2017/095241 A2, titled “Flow Path Sensing for Flow Therapy Apparatus”, filed on Dec. 2, 2016, and International Application No. WO 2016/207838A1, titled “Breathing Assistance Apparatus”, filed on Jun. 24, 2016, which are hereby incorporated by reference in their entireties.

The various configurations described are exemplary configurations only. Any one or more features from any of the configurations may be used in combination with any one or more features from any of the other configurations.

A respiratory apparatus, such as breathing assistance apparatus 10 of FIGS. 1, 2, and 2A-2B, can include any number of features including those explicitly numerated herein as well as those features known to those of skill in the art. One example respiratory apparatus which is commercially available from Fisher & Paykel Healthcare of Auckland, NZ is the Airvo 3. It is to be understood, however, that other respiratory apparatuses can benefit from the disclosure herein.

### Training/Simulation Needs

In learning to use the respiratory apparatus disclosed herein, operators need to be able to train in a safe and practical way. This disclosure includes systems and methods on how to train operators through the use of simulation. Simulation can involve the simulation of a patient connected to the respiratory apparatus, and/or simulation of low-pressure and/or high-pressure oxygen, or other therapeutic gases or gases mixtures being delivered using the respiratory apparatus. The training system simulations can enable safe training-potentially in a constrained environment—of operators without the need to train on actual patients or use real, concentrated oxygen (O₂). These simulations allow operators to train in a safe manner, avoiding any risk of injury to patients or potential mishaps with O₂ and its storage. For example, highly-concentrated and/or pressurized O₂ can be combustible. Furthermore, it may be undesirable to expend O₂ during training exercises in general or in certain contexts (for example, in a field situation where O₂ availability may be rationed or otherwise limited). To provide the simulation/training experiences, the respiratory apparatus can be designed to operate using actual operating parameters. For example, the respiratory apparatus can be configured to operate according to a specified flow rate, temperature, humidity level, or any other therapy parameter, in order to emulate how it may operate when in use with an actual patient. Although actual oxygen is not provided to the apparatus, the operator can interact with the apparatus in the same way as if actual oxygen were provided. The training system can thus enable operators to get a true sense of how the respiratory apparatus works without jeopardizing a patient or an operator.

The training system simulations can involve the use of peripheral devices. A peripheral device can be a mobile device, tablet, phone, or any other programmable hardware that can be configured to communicate with a respiratory apparatus and simulate patient responses and/or supplementary gases signals. The peripheral device can effectively act as a virtual patient, receiving data from the respiratory apparatus (e.g., flow rate or FiO₂ setting) and transmitting data in response to received data and a patient model (e.g., a simulated patient SpO₂). For example, the peripheral device could be a smartphone executing a software application, allowing the smartphone to serve as a peripheral device and to communicate with a respiratory apparatus and simulate patient responses and/or supplementary gases signals. The simulated patient responses can include a simulated patient SpO₂, respiratory rate, other parameters such as minute ventilation, tidal volume, peak inspiratory demand, etc.

The use of peripheral devices for simulation also enables training in any location and can allow training to be mobile, that is, allowing training to be remote. With peripheral devices designed to simulate use of the respiratory apparatus, operators can train without needing to be near a gases source or patient. Operators and/or users can use the simulation to train such that the operator becomes familiar with the use of all operational parameters and understands alarms by experience as opposed to sitting through a presentation, video, or demonstration of the respiratory apparatus.

Furthermore, with a respiration apparatus that can provide multiple types of therapies (including, for example, high flow therapy and CPAP, BiPAP (NIV), or BCPAP), demonstrations of the actual operation of the respiration apparatus can be very challenging and dangerous. Certain therapies may have lower oxygen (FiO₂) requirements to maintain the same SpO₂ target in a patient, when compared to high flow therapy. For example, CPAP, BiPAP, and/or BCPAP may require a lower percentage FiO₂ than high flow therapy to maintain patient blood oxygenation at safe levels. The simulated patient model or virtual patient can account for the different oxygen requirements of these different therapies. The simulated patient model can account for the differences by adjusting, scaling, and/or modifying model parameters and the like depending on the therapy model configured on the apparatus. For example, the patient model may include a component that models an SpO₂/FiO₂ ratio. This ratio represents how efficiently the oxygen received from the respiratory apparatus becomes bound to hemoglobin in the blood of a patient. This component could be adjusted such that for a given FiO₂ setting on the respiratory apparatus, the simulated patient SpO₂ will be higher or lower. This adjustment can be dependent on the therapy mode. In an NIV mode or other pressure-based therapy modes, a lower FiO₂ may be required to achieve a target SpO₂. Conversely, in high flow therapy mode(s), higher FiO₂ may be required to achieve the same target SpO₂. With the modeling of the SpO₂/FiO₂ ratio, a trainee can experience switching between therapy modes/types on an apparatus capable of providing multiple therapy types and how the FiO₂ requirements can be adjusted to maintain patient SpO₂.

Without simulation capabilities, a demonstration may require both a patient be connected to the apparatus to receive therapy, and the use of concentrated O₂ (for example, 90-100% O₂), at high pressures. Training in this condition can be unnecessarily dangerous to the patient and operator due to the use of O₂ by an inexperienced operator, which is combustible and/or stored under high pressure conditions. The present disclosure can allow a safe, but full hands-on experience of how the respiratory apparatus operates and allows operators to learn in a safe environment.

Simulation can also allow operators to provide maintenance services and test the full suite of functionality on the respiratory apparatus without the need to connect to pressurized and concentrated O₂ or couple the respiratory apparatus to a live patient. Maintenance procedures may be improved by being able to safely test certain features of the respiratory apparatus with a simulated oxygen source and/or patient. For example, the respiratory apparatus may have audio-visual alarms relating to the SpO₂ levels of a connected patient. It may be desirable to artificially create a situation wherein a virtual patient is desaturating (that is, experiencing a precipitous SpO₂ decline) to verify an alarm triggers correctly. Without a virtual patient, such alarms may be difficult to trigger in a real-life scenario using a real patient. Simulated patient responses may be similarly useful for other alarms, for example, respiratory rate or other breathing-related alarms. The peripheral device can simulate a patient and supplementary gases using a virtual patient model, which enables maintenance technicians to test and maintain the respiratory apparatus using the peripheral device.

Respiratory apparatuses are often equipped with software and electronic hardware that facilitates electronic communication with one or more auxiliary devices, including, for example, a pulse oximeter. Auxiliary devices can be configured for measurement of conditions. Auxiliary devices can be configured to communicate with the respiratory apparatus and/or the peripheral device via a wired or wireless connection. For example, a wired connection can be via a USB (Universal Serial Bus) interface or other ports or connectors through which an auxiliary device can be mechanically and electrically connected to the respiratory apparatus and/or the peripheral device, for example, using a cable. In the case where an auxiliary device is a pulse oximeter, the respiratory apparatus can receive measurements from the pulse oximeter and can present the received measurements on a display screen, such as, for example, an LED touch display screen (which can be the user interface 54 of FIGS. 1, 2, 2A-2B). In the training system disclosed herein, it may not be necessary to connect to a pulse oximeter. Instead, a custom protocol can be used to perform communication between any or all of the respiratory apparatus, the peripheral device, and the auxiliary device (for example, the pulse oximeter). The peripheral device can emulate pulse oximeter readings using the custom protocol such that the respiratory apparatus would treat the emulated pulse oximeter readings as if they were provided by a pulse oximeter taking contemporaneous measurements from a live patient connected to the respiratory apparatus.

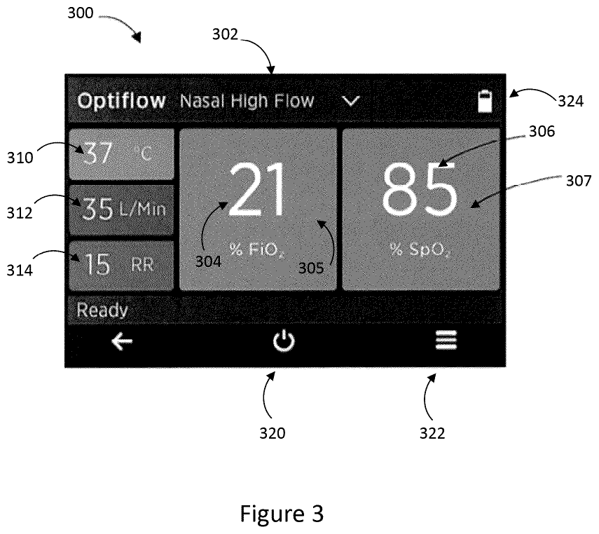

An example graphical user interface 301 is shown in FIG. 3. FIG. 3 illustrates an example touch screen display with various parameters displayed including temperature 311, flow rate 312, respiration rate 314, FiO₂ 304, SpO₂ 306, battery level 325, flow therapy mode 302, power 320 and menu options 322. Other parameters may be contemplated. For example, other breathing-related parameters may be displayed, e.g., minute ventilation, tidal volume, peak inspiratory demand, etc. The parameters displayed may be dependent on the type of therapy being provided by the respiratory apparatus. For example, if the respiratory apparatus is in CPAP or NIV therapy mode, parameters such as PEEP, IPAP and/or EPAP may be displayed instead of or in addition to other parameters such as flow rate. Each displayed item can be a software button that can be touched by a user to bring up a submenu that allows the user to adjust operation of the respiratory apparatus. For example, a first parameter display element 305 and a second parameter display element 307 can be selected and changed to output a different parameter other than FiO₂ and SpO₂.

For example, an HPO source may be connected, and the operator may select a percentage FiO₂ and a particular flow rate for the respiratory apparatus to deliver via the graphical user interface 301. This feature and others are described in International Application No. WO 2019/112447 A1, which is incorporated herein in its entirety by reference and should be considered part of the disclosure.

Using data received from an auxiliary device (for example, a connected pulse oximeter), the respiratory apparatus can display the patient's (peripheral) blood oxygen saturation (SpO₂). This provides an operator with visual feedback on the effects of the current therapy and therapy parameter settings (for example, flow rate or pressure, and FiO₂). Alternatively, the respiratory apparatus may have built-in sensors that allow the respiratory apparatus to gather information and data, such as the patient's blood oxygen saturation. The data may also be used as part of a closed-loop oxygen control scheme such as those described above and disclosed in International Application Nos. WO 2019/070136 A1 and WO 2021/049954 A1, the entirety of each of which are incorporated by reference herein and should be considered part of the disclosure.

With these features in mind, the present disclosure includes a description of a peripheral device with which the presence of a live patient connected to the respiratory apparatus can be simulated. The simulation can be achieved by the peripheral device emulating readings of a pulse oximeter connected to an imaginary patient. Although the present disclosure provides a peripheral device, it is to be understood that the functions performed by the peripheral device can also be incorporated in software embedded in the respiratory apparatus itself and a simulation mode on the respiratory apparatus itself can be used to provide the training and experimentation described herein.

### Peripheral Device

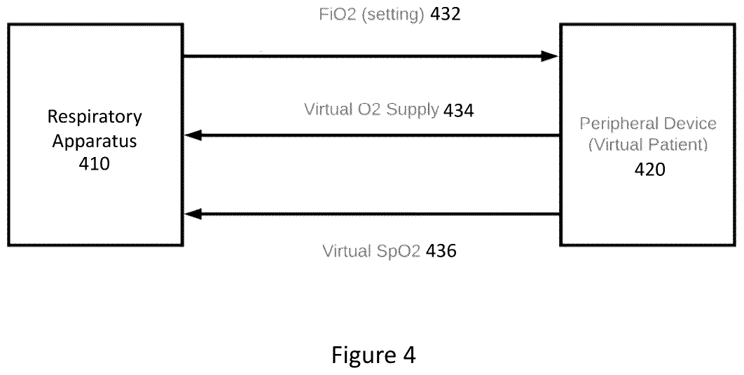

FIG. 4 illustrates a schematic of a peripheral device 420 connected and communicating with a respiratory apparatus 410. The respiratory apparatus 410 can have one or more interface ports. The peripheral device 420 can be connected to the respiratory apparatus 410 via the one or more interface ports. Through the one or more interface ports, the respiratory apparatus 410 can communicate the FiO₂ setting 432 (and additionally or alternatively, the flow rate setting) specified by the operator to the peripheral device 420. This way, the peripheral device 420 can understand what virtual concentration of oxygen is being “provided” to the virtual patient. Through the one or more interface ports, the peripheral device 420 can communicate the virtual O₂ supply 434 and the virtual SpO₂ 436 to the respiratory apparatus 410.

The peripheral device 420 (also referred to herein as a “peripheral simulation device”) may be a tablet, laptop, smart phone or any other device suitable for executing a software program. The operating system of the peripheral device 420 may be Windows, MacOS, Android, IOS, a Unix or Linux-based operating system, or any other suitable operating system. The peripheral device 420 can have one or more interface ports.

The interface ports on the peripheral device 420 and the respiratory apparatus 410 can be wired or wireless. For example, one of the interface ports of either the peripheral device 420 or the respiratory apparatus 410 can be a USB port, although other physical connector port types can be used (e.g., an 8-pin RJ45 ethernet connector, RS-232 9-pin connector, etc.). Appropriate wired protocols such as USB, 12C, SPI, etc. can be used. . . . Alternatively, Wi-Fi, Bluetooth, NFC, or similar wireless protocols could be used to connect with an interface port of the peripheral device 420.

As mentioned above, the peripheral device 420 may be capable of communicating with the respiratory apparatus 410 using a recognised protocol while connected via one or more interface ports. An example respiratory apparatus 410 can have two USB interfaces installed, which are intended for auxiliary devices, including patient monitoring equipment, but are also suitable for a peripheral device 420 emulating the auxiliary devices. Thus, the peripheral device 420 may communicate virtual SpO₂ data, plethysmography waveforms, respiratory rate, or potentially any other relevant patient physiological parameters. This effectively creates a “virtual patient” and can also be able to simulate a virtual patient connected to an auxiliary device such as a pulse oximeter.

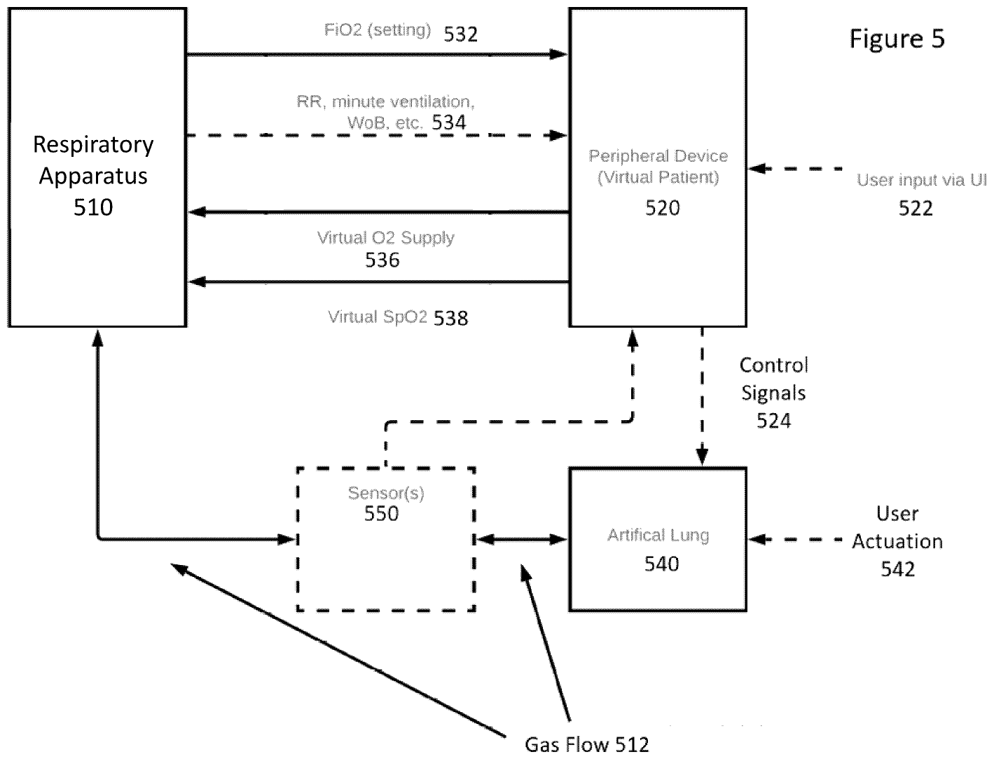

FIG. 5 illustrates a schematic of a peripheral device 520 connected and communicating with a respiratory apparatus 510, sensor(s) 550 (for example, a flow and/or pressure sensor) and another example auxiliary device, an artificial lung 540. The respiratory apparatus 510 can have one or more interface ports. The peripheral device 520 can be connected to the respiratory apparatus 510 via the one or more interface ports. Through the one or more interface ports, the respiratory apparatus can communicate the FiO₂ setting 532 to the peripheral device 520. Through the one or more interface ports, the respiratory apparatus 510 can also communicate advanced settings or calculations 534 (for example, respiration rate, minute ventilation, work of breathing (WoB), and the like) to the peripheral device 520. Through the one or more interface ports, the peripheral device 420 can communicate the virtual O₂ supply 536 and the virtual SpO₂ 538 to the respiratory apparatus 510.

The user, or operator, of the peripheral device 520 can give user input via a UI 522 to the peripheral device 520.

The peripheral device 520 can also be used as part of the system to provide any forms of virtual or emulated patient feedback from any number of auxiliary devices. With reference to FIG. 5, an example auxiliary device can be an artificial lung 540. Other auxiliary devices can include a test lung, spirometers, artificial lungs, or any other equipment that can be used to emulate human lung mechanics. Auxiliary devices can be connected to the respiratory apparatus 510 and be controlled by a peripheral device 520 to respond to the respiratory apparatus output (for example, gases flow 512, whether simulated from the artificial patient exhalation or an actual gases flow from the respiratory apparatus 510) appropriately. This could enable the respiratory apparatus itself, via sensor measurements, or the peripheral device software, to generate training data relating to respiratory rate, minute volume/ventilation, inspiratory and expiratory times, and the like.

The sensor(s) 550 are optional as the respiratory apparatus 510 can have sensors for monitoring conditions of the simulated patient. The sensor(s) 550 or the respiratory apparatus 510 may run respiration rate algorithms and/or gather other important information about the patient, virtual patient, conditions in the environment, or virtual conditions in the environment. The sensor(s) 550 can provide this information to the peripheral device 520 and/or to the respiratory apparatus 510.

### Peripheral Device Software & Patient Models

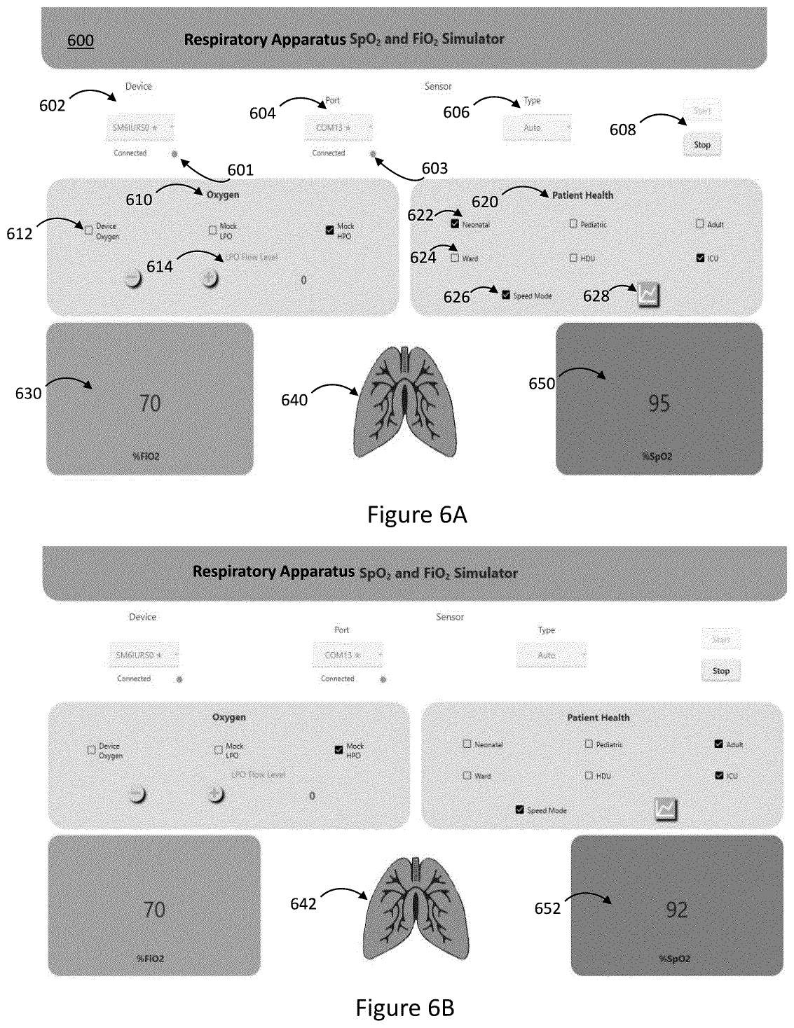

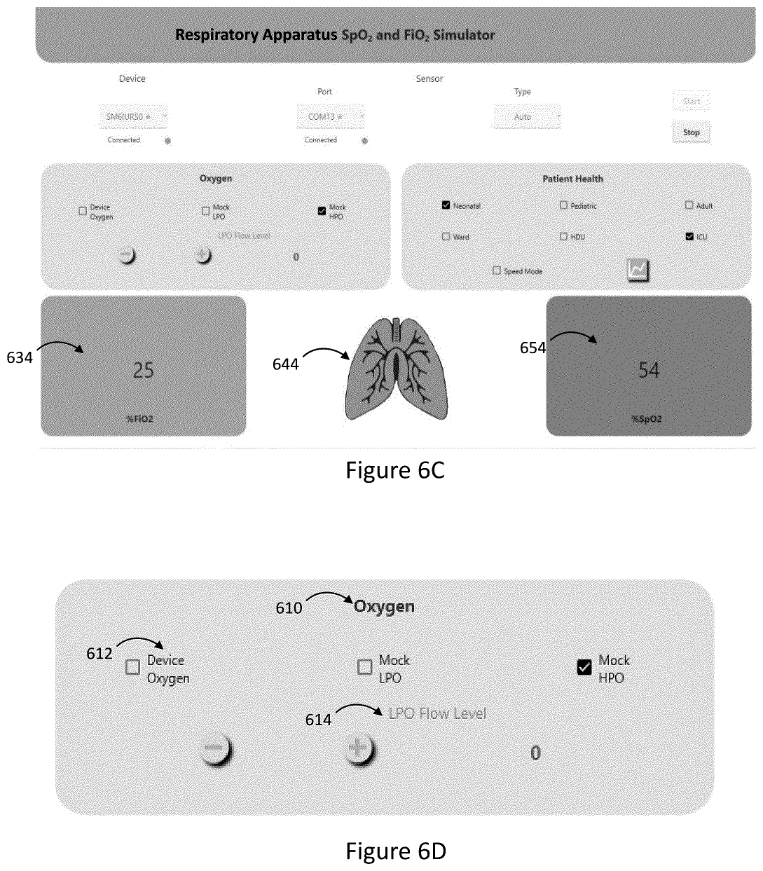

FIG. 6A shows an example graphic user interface 600 (which can be referred to as interface 600) for the peripheral device (for example, the peripheral device 420 of FIG. 4).

In some implementations, the interface 600 can show what respiratory apparatus (for example, the respiratory apparatus 410 of FIG. 4) is connected to the peripheral device via the connected device element 602. Information about the connected respiratory apparatus displayed may entail showing a device model name or more specific information, such as the device identifier, serial number, etc. The user can additionally or alternatively use the connected device element 602 to choose which respiratory apparatus the peripheral device is connected to. The respiratory apparatus can be chosen via a drop-down menu 602 in FIG. 6A. However, it is understood that any adequate way of choosing an option in a user interface would be suitable, such as radio buttons, a searchable list, a table, or the like. Additionally, the connected device element 602 can show whether the peripheral device has connected or failed to connect to the respiratory apparatus. For example, the connected device element 602 may have a first indicator 601 to demonstrate that a connection has been established between the peripheral device and the respiratory apparatus, and a second indicator to demonstrate that a connection has not been established. For example, a green indicator 601 can be used to demonstrate that a connection has been established between the peripheral device and the respiratory apparatus, and a red indicator can be used to demonstrate that a connection has not been established.

In some implementations, the interface 600 can show what peripheral device is connected via the connected device element 602 to an auxiliary device. Examples of the auxiliary device can include the sensors 550 and/or an artificial lung 540 in FIG. 5. In some implementations, the connected device element 602 can indicate that the peripheral device is connected to one or more devices wherein the devices are either respiratory apparatuses or auxiliary devices.

The interface 600 can show a port involved in the connection between the peripheral device and the respiratory apparatus via the port connection element 604. In some implementations, the port connection element 604 can show the port of the peripheral device to which a respiratory apparatus or an auxiliary device is connected. In some implementations, the port connection element 604 can show the port of the respiratory apparatus to which the peripheral device is connected. In some implementations, the port connection element 604 is labelled as either a respiratory apparatus port, a peripheral device port, or an auxiliary device port. In some implementations, the port connection element 604 will correspond to the connected device element 602 such that the device and port are matched. In some implementations, if there are multiple connected devices to the peripheral device, the connected device element 602 will have multiple entries which also correspond to multiple entries in the port connection element 604. Additionally, the port connection element 604 can show whether the peripheral device has connected or failed to connect to the respiratory apparatus. For example, the port connection element 604 may have a first indicator 603 to demonstrate that a connection has been established between the peripheral device and the respiratory apparatus, and a second indicator to demonstrate that a connection has not been established. For example, a green indicator can be used to demonstrate that a connection has been established between the peripheral device and the respiratory apparatus, and a red indicator can be used to demonstrate that a connection has not been established.

The interface 600 can also have a type indicator element 606. In some implementations, the type indicator element 606 indicates the type of sensor or other auxiliary device being used to gather data. In some implementations, the type indicator element 606 indicates the way in which a sensor gathers data. For example, the type indicator element 606 could be set to “auto” to automatically record data. In some implementations, the type indicator element 606 could indicate the type of simulation that will be run. For example, the type indicator element 606 could be set to “auto” to automatically run the simulation in real-time. Alternatively, the type indicator element 606 could be set to “step” which allows a user to make incremental steps of time through a simulation. Real-time simulation may be beneficial to practical training with the respiratory apparatus—for example, how to respond to a desaturating patient. Stepped simulation can have other benefits—for example, developing understanding or comprehension of the operations of the respiratory apparatus by slowly stepping through time and studying changes in the virtual patient's parameters.

The interface 600 can also have a start/stop element 608. The start/stop element 608 allows the user to start or stop the simulation. In some implementations, the start/stop element 608 can form an on/off or connect/disconnect switch. When the peripheral device is set to stopped or ‘disconnected’, the respiratory apparatus can present alerts that it cannot detect a pulse oximeter. These alerts may be representative of the alert that appears when an actual pulse oximeter is disconnected or dislodged such that the connection is interrupted.

The start/stop element 608 can be used to provide authentic training experiences. For example, the peripheral device could be providing “virtual oxygen” before an operator attempts to switch the respiratory apparatus off. The respiratory apparatus will prevent the respiratory apparatus from being turned off when the “virtual oxygen” is still connected and display on its interface a warning alert that O₂ must be disconnected before the respiratory apparatus can be safely turned off. In another example of a training experience, if attempting to turn off the respiratory apparatus while operating in HPO mode, the operator may first need to reduce the FiO₂ setting to a minimum (20.9 or 21%-nominal ambient air O₂ concentration), which will cause the peripheral device to stop providing virtual oxygen, allowing the respiratory apparatus to be turned off. In another example of a training experience, if attempting to turn off the respiratory apparatus while operating in LPO mode, the operator may first need to reduce the virtual LPO flow to zero using the peripheral device software application interface element(s). This will cause the peripheral device to cease delivery of virtual oxygen. In both of these situations, these alerts and training experiences represent simulations of the steps a user of the respiratory apparatus disclosed herein may need to carry out during ordinary operation of the respiratory apparatus (except with real oxygen sources) to safely power down the respiratory apparatus when actually using the respiratory apparatus to treat a patient.

As shown in FIG. 6D, the interface 600 can also have an oxygen indicator element 610. The oxygen indicator element 610 has a gases type indicator element 612 and a titration element 614. The peripheral device software is programmed to simulate the conditions that would indicate the gases type (for example, the presence of LPO or HPO or the use of ambient air processed by the respiratory apparatus) when the operator selects the gases type via the gases type indicator element 612 on the peripheral device user interface 600. When LPO has been selected, the peripheral device software application allows titration of oxygen via the titration element 614 of the peripheral device user interface 600, simulating what a user of the respiratory apparatus disclosed herein may do with a physical blender (such as a rotameter).

The interface 600 can also have a patient health element 620. The patient health element 620 can have a model choice element 622, a severity choice element 624. a speed mode element 626, and an output graph element 628.

Regarding the model choice element 622, a variety of patient models can be chosen by the user of the peripheral device. The models can incorporate learnings from a variety of data sources, including results from clinical trials and studies. Mathematical approximations can be used in the construction of the models where empirical data for certain types of patients are unavailable or insufficient. For example, differences in values of parameters between a pediatric patient model and an adult patient model may be determined based on a linear scaling factor applied to empirical data available from group(s) of either type of patient. Alternatively, more complex relationships might be used for more accurate representation of differences between patient types where sufficient data is not available for one or more groups.

In the example interface 600, three types of patient models are provided by the model choice element 622, namely neonatal, pediatric, and adult. These may be selected using the peripheral device software application user interface and will determine the data communicated to the respiratory apparatus when connected. In an example, for specific FiO₂ and flow rate settings on the respiratory apparatus, the neonatal model may output SpO₂ data which indicates the FiO₂ and flow rate settings are suitable (for example, SpO₂ is above 96%). For these same settings, the adult or even the paediatric models may output SpO₂ data which instead indicates the settings are not suitable for the patient (for example, SpO₂ is below 95%).

The different types of patient models may be related by linear factor. For example, the adult model may be equivalent in structure to the paediatric model, but adjusted by a linear scaling factor. Additionally or alternatively, the models may be calibrated based on certain inputs. For example, there may be a universal model in which a user can specify a patient's height, weight, ideal body weight (IBW) or other equivalent size parameter, and the training software will scale the model appropriately. As therapy parameters, such as the flow rate setting, are very closely related to a patient's size, this approach may enable more accurate training experiences.

However, any number of patient models can be provided by the model choice element 622. For example, the model choice element 622 could have a dropdown menu of model choices or allow a user to type in part or all of the name of a model (e.g., via a search function) to choose a model.

The patient models may optionally be further specified by the severity of their conditions via the severity choice element 624. For example, FIG. 6A illustrates that an operator may specify an indicator of severity such as the patient is in an intensive care unit (ICU), high dependency unit (HDU), or a regular ward. By enabling one of these severity specifiers, additional parameters, constants or other model features may be applied to a patient model. For example, the model may be scaled linearly when one of the severity specifiers is selected. Enabling certain severity specifiers can result in higher FiO₂ requirements to maintain a healthy (virtual) SpO₂, representative of a patient in a more severe state. Conversely, enabling certain severity specifiers can result in a lower FiO₂ requirements to maintain a healthy (virtual) SpO₂, representative of a patient in a less severe state. Alternatively, existing parameters, constants and/or the like in a patient model may be modified directly. Regardless of the precise implementation, these changes will affect the output of the patient model(s) depending on the operator's selection.

Referring to the example in FIG. 6B, the adult patient model outputs SpO₂ data which indicates the settings are insufficient for the patient (for example, SpO₂ is below 95%), since it is specified in this training example that the adult is in an ICU. If the “ICU” specifier is switched to “Ward”, the model will output SpO₂ that indicates the settings are sufficient for the patient. These changes in the setting reflect the severity conditions or statuses. An actual ICU patient is likely to suffering from worse respiratory insufficiency (e.g., due to a more severe respiratory condition, ailment, or injury) and therefore needs more respiratory support. This means that flow and/or FdO₂ or FiO₂ settings may need to be increased. Similarly, an HDU patient may also need more respiratory support, though not to the extent an ICU patient would. In comparison, a regular ward patient may require a lesser degree of respiratory support. By allowing specification of the virtual patient's condition in this way, an operator can experience adjusting the respiratory therapy device settings across a broad spectrum.

In some implementations, the user can modify the patient model via the severity choice element 624 based on specification of the patient's condition(s). In this example, the severity choice element 624 can include choices specifying that the virtual patient has Stage I, II, III or IV chronic obstructive pulmonary disease (COPD), for example. In general, other respiratory conditions or ailments could be used. Some choices for the severity choice element 624 may include both an indicator of severity (for example, the patient is in an ICU) and other patient conditions (for example, having Stage II COPD).

Software running on a hardware processor in the peripheral device can have a variety of mathematical patient models embedded within it to provide the user with a myriad of simulations on which to practice. These models have different parameters and constants depending on what type of patient, disease type or condition, or situation they represent.

As shown in FIGS. 6A-6C, the interface 600 can have a FiO₂ indicator element 630. This can show the user the FiO₂ levels that was set on the respiratory apparatus. This reading can help a maintenance worker check if the respiratory apparatus is providing the correct FiO₂ in the gases mixture.

The interface 600 can also have a lung diagram element 640. The color of the lung diagram element 640 in the image can vary based on the virtual SpO₂ reading. For example, in FIG. 6A, the lung 640 may be almost entirely green and optionally have a small amount of red for example at the top portion of the lung 640, representing a positive SpO₂ reading. In FIG. 6B, the SpO₂ reading is less satisfactory, and the lung 642 is consequently significantly less green-colored, and optionally have an increased amount of red as compared to FIG. 6A for example at a top portion of the lung 640. FIG. 6C illustrates an extreme situation where the delivered FiO₂ and/or flow result in a severe virtual SpO₂ reading and thus the lung 644 could be almost entirely red. The lung diagram element 640 can show a more detailed image of a lung. For example, a 2-D or 3-D model of a lung may instead be used to provide further visual feedback to an operator.

The peripheral device can have a speed mode which can be activated by interacting with a speed mode element 626. Speed Mode applies an acceleration (or speeding up) factor to the virtual patient model, causing more rapid responses to changes in respiratory apparatus output settings or the virtual patient model (for example, switching from “Ward” to “ICU”). This is useful where an operator may not be interested in studying a realistic response, but rather wants to rapidly test changes in a patient's parameters during respiratory therapy.

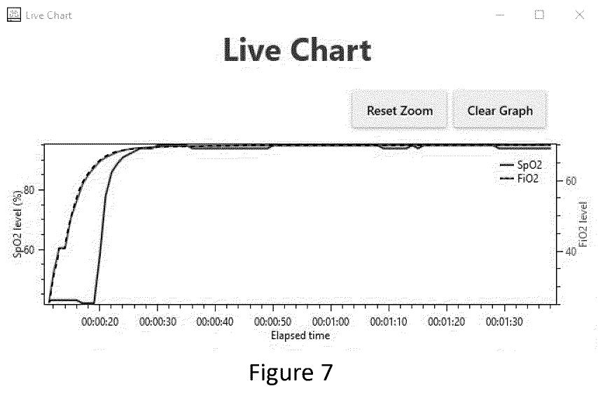

Via the output graph element 628 (for example, when the user selects the output graph element 628), the peripheral device may optionally display (for example, in a pop-up window or otherwise) real-time simulated data to an operator in the user interface. The data display may have a variety of embodiments and may be configurable based on operator preferences. For example, different chart/graph types may be used, axes scales may be manually or automatically adjusted, colours (background, data/legend, labels) may be adjusted. An example output graph is illustrated in FIG. 7.

The interface 600 can also have a SpO₂ indicator element 650. This can show the user what the SpO₂ levels that result from the specific inputs to the respiratory apparatus and the particular conditions (for example, the model choice via the model choice element 622 and the severity choice via the severity choice element 624).

FIGS. 6A through 6C show example interface 600 settings, inputs, and outputs. FIG. 6A shows that a sample interface where the FiO₂ from the respiratory apparatus is 70% and the flow rate is 20 LPM (not shown) and the patient model is Neonatal-ICU. Here, an example virtual SpO₂ reading 650 is 95%.

FIG. 6B shows that a sample interface where the FiO₂ from the respiratory apparatus is 70% and the flow rate is 20 LPM (not shown), and the patient model is Adult-ICU. Here, an example virtual SpO₂ reading 652 is 92%.

FIG. 6C shows that a sample interface where the FiO₂ from the respiratory apparatus is 25% and the flow rate is 20 LPM (not shown) and the patient model is Neonatal-ICU. Here, an example virtual SpO₂ reading 654 is 54%.

### Virtual Oxygen Adjustment

As a specific example of an implementation of the present disclosure, adjustment of FiO₂ in the system can depend on whether LPO or HPO has been specified. In both instances, the peripheral device software application will simulate the presence of either source type, creating a “virtual oxygen source”. As described above, the respiratory apparatus can receive supplemental gases including LPO and HPO. Because HPO source can be connected via a proportional valve, which can be electronically controlled by the respiratory apparatus to control the flow rate of oxygen from the HPO source, when HPO has been selected via the user interface of the peripheral device, an operator can control (titrate) the FiO₂ on the respiratory apparatus interface.

This feature and others are described in International Application No. WO 2019/112447 A1. FIG. 3 is extracted from the WO 2019/112447 A1 publication and shows an example user interface. International Application No. WO 2021/048744 A1, which is incorporated herein by reference in its entirety and should be considered part of the disclosure, describes how the respiratory apparatus can detect which type of supplementary oxygen source is detected.

### Training/Demonstration Mode

The training system can use a respiratory apparatus that is solely for training. Alternatively, the training system can use respiratory apparatus that is used for providing therapy to patients, but also have a training/demonstration/simulator mode. Having a training/demonstration mode increases the safety of operation of a respiratory apparatus. Users are able to understand and simulate situations for proper operation of the respiratory apparatus prior to treating patients. Similarly, users are less likely to train to operate the respiratory apparatus while simultaneously giving treatment of patients. When treating a patient, it is critical that actual patient readings are captured for monitoring purposes; real oxygen may need to be delivered and controlled appropriately.

The training/demonstration/simulator mode for the respiratory apparatus allows for interaction with virtual patients and virtual oxygen sources. Such a training mode may only be accessible on certain marked respiratory apparatuses. A marked respiratory apparatus may have a software key programmed to a computer memory location within a non-transitory storage medium during manufacturing which allows a respiratory apparatus to operate in training mode when configured to do so (for example, by selecting such a mode on the respiratory apparatus user interface). Marked respiratory apparatuses may only be able to operate in training mode or only in the standard suite of modes.

Respiratory apparatuses with training mode software keys can have certain therapy features locked out, or certain control features to prevent use with actual patients entirely. For example, if a patient is detected using sensors, algorithms, or other means, the respiratory apparatus may cease operations and present warnings that it is a training-only respiratory apparatus or currently intended to be for training only.

Respiratory apparatuses with training mode software keys can deliver therapy to patients if configured to do so. This may involve entry of a specific password, PIN, touch sequence, or other verification procedure to enable switching from training mode to normal operations mode where the full suite of modes are available. Additionally, or alternatively, a physical key can be used to switch a respiratory apparatus out of training/demonstration mode.

As an alternative to a software or hardware key, marked respiratory apparatuses may be respiratory apparatuses specifically manufactured for training and demonstration purposes only. This means certain respiratory apparatuses may have elements removed or added to prevent their use as patient treatment respiratory apparatuses. For example, humidification chamber receptacles may be covered or sealed-off to discourage use of these respiratory apparatuses for therapy.

Additionally or alternatively, a chamber-off detection algorithm could be periodically executed when the respiratory apparatus is in the training mode. The chamber-off detection algorithm can verify that no humidification chamber is connected. If a humidification chamber is detected, the respiratory apparatus may cease providing flow and/or otherwise prevent further use of the respiratory apparatus.

Examples of a leak detection process are described below that can be used in the training mode disclosed herein. FIG. 9 illustrates an example flow diagram of a leak detection process or algorithm 700. The leak detection algorithm may start 701 operation immediately upon commencement of normal operation of the apparatus (which may include a training mode) or after a preconfigured delay period to allow the operating characteristics time to settle. After starting, the leak detection algorithm continuously operates at a first-stage leak evaluation 702 during normal operation of the apparatus. In this first-stage leak evaluation 702, the pressure variable from a pressure sensor sensing the flow of gases in the flow path is compared against a definitive leak threshold and a possible leak threshold to determine if one or more conditions are satisfied.

If the pressure variable is below the definitive leak threshold at the first-stage leak evaluation 702, a leak detected condition is satisfied 703 and the process moves to a leak detected state or stage 704 in which a leak alarm is generated. During this alarm state 704, the algorithm can be configured to continually or periodically monitor the pressure variable of the flow of gases to check for whether the leak (e.g. chamber off) has been resolved. If the pressure variable is above the threshold, a leak resolved condition is satisfied 711 and the algorithm exits the leak detected state 704 back to the first-stage leak evaluation 702 and normal operation.

If the pressure variable is below the possible leak threshold at the first-stage leak evaluation 702, a possible leak condition or state 706 is satisfied, and the process moves to a second-stage leak evaluation 707. The purpose of the second-stage leak evaluation 707 is to either confirm the possible leak condition as a definite leak, or discard the possible leak condition as being no leak.

The second-stage leak evaluation 707 can include increasing the current operating motor speed by a dynamic or predetermined increment to a higher motor speed, and then re-assessing the updated pressure variable at the higher motor speed to the definite leak threshold and possible leak threshold to resolve the possible leak condition as either a definite leak or no leak. If the re-assessment or evaluation at the higher motor speed does not resolve the possible leak condition in the second-stage leak evaluation 707, the motor speed is incremented again to yet another higher motor speed. In the second-stage leak evaluation 707, the possible leak condition is resolved as a definite leak 708 if the updated pressure variable at the higher motor speed is below the definite leak threshold, and the algorithm exits the second-stage leak evaluation 707 to the leak detected state 704. If a no leak condition is satisfied 709, the algorithm exits to the first-stage leak evaluation 702 and normal operation. Optionally, upon exiting the second-stage leak evaluation 707 with no leak detected 709, the algorithm may be configured to update or adjust one or more of the threshold limits at 712.

If neither of the above leak or possible leak conditions are satisfied, the pressure variable to the definite and possible leak thresholds, the first-stage leak evaluation 702 considers that no leak is detected 705 and the algorithm remains in the first-stage leak evaluation.

The chamber-off or leak detection processes disclosed herein or any other suitable leak detection process can be used when training an operator on the use of the respiratory apparatus to verify whether there is a “leak”. If no “leak” is detected, a humidification chamber is considered to be connected, which may be undesirable as the training-only respiratory apparatus or a respiratory apparatus in the training mode may be inadvertently misused for providing therapy to a real patient.

### Additional Features

The simulation/training mode may be accompanied by hardware components to create a simulation/training system. The simulation/training system can use an “artificial finger” which synthesizes pulse oximetry light signals from a finger to be used with a real pulse oximetry device. The artificial finger could be an auxiliary device connected to and operating in conjunction with the peripheral device. For example, light signals can be generated according to control signals issued from the peripheral device, which has the virtual patient models embedded, to the artificial finger. Alternatively, the artificial finger can be implemented in software on the peripheral device, by use of the microcontroller/processor, memory, storage, and virtual patient models on the peripheral device.

A remote user interface device (for example, a touch screen) might accompany the artificial finger to a convenient display for feedback and interaction.

The simulation/training system could have other physical apparatuses to help develop familiarity with certain aspects of the respiratory apparatus set-up process and operation. In some implementations, the simulation/training system may include imitation LPO and HPO ports to electronically connect (for example, by wired or by wireless connections) to the peripheral device. The imitation LPO and HPO ports can be similar in appearance and structure to those disposed on the respiratory apparatus, but instead the imitation LPO and HPO ports can be in electronic communication with a peripheral device. An operator could physically connect an LPO or HPO connector to the imitation LPO and HPO ports such that sensors embedded within or fixed to the imitation LPO and HPO ports would detect a successful connection, and send a signal to a peripheral device running the software application. In some implementations, the LPO or HPO connectors could be imitation LOP or HPO connectors. After connecting the connectors to the ports, the peripheral device can “unlock” control of the virtual oxygen source for the operator to use. By incorporating hardware components that simulate setting up the respiratory apparatus, a more effective and representative simulation/training experience may be provided as compared to selecting the supplementary oxygen source type on a digital interface. This may help users develop familiarity with the different port types and their significance.

In some implementations, an imitation low-pressure oxygen flow meter could be electrically connected to the peripheral device for training/simulation instead of using the digital interface on the peripheral device. For example, an imitation flow meter may be in electronic communication with a peripheral device, sending signals to the device based on adjustments made on the imitation flow meter. These signals would trigger the peripheral device to make appropriate adjustments to the virtual LPO source.

In some implementations, an artificial lung and/or an actual oxygen source can be used to emulate some patient physiological responses. This may help users to develop insight on mechanical lung response to the respiratory apparatus flow output while using the simulation/training system.

In some implementations, the peripheral device software could be given some control over the respiratory apparatus (as opposed to passively serving as a virtual patient/configurable patient model) and some or all settings of the respiratory apparatus could be adjusted on the peripheral device.

The peripheral device can also be used as a training tool to teach care providers how to react under specific patient treatment scenarios. For example, the system can simulate a patient having respiratory distress, requiring a care provider to react by adjusting certain parameters on the respiratory apparatus.

The peripheral device can be used with the humidifier of FIGS. 2A and 2B in order to simulate the oxygen usage of a patient. The humidifier 2A and 2B comprises a communication module e.g. Bluetooth, Wifi and a cellular communication module that can be used to communicate with the peripheral device. The humidifier 10 can also communicate with a flow generator e.g. a ventilator and change settings of the ventilator based on the output of the patient simulator executing in the peripheral device. The humidifier 10 may be configured to control the operation of the ventilator and simulate delivery of oxygen to a patient. The humidifier 10 may be configured to control in real time operation of the ventilator e.g. controlling an O₂ valve of a ventilator and flow rate or pressure delivered by the ventilator. The advantage of the training system (the simulation system) described herein is that it simulates a patient's behaviour without the need for delivering O₂ to a patient or having O₂ flowing through the respiratory apparatus.

### Terminology

Although this disclosure has been described in the context of certain embodiments and examples, it will be understood by those skilled in the art that the disclosure extends beyond the specifically disclosed embodiments to other alternative embodiments and/or uses and obvious modifications and equivalents thereof. In addition, while several variations of the embodiments of the disclosure have been shown and described in detail, other modifications, which are within the scope of this disclosure, will be readily apparent to those of skill in the art. It is also contemplated that various combinations or sub-combinations of the specific features and aspects of the embodiments may be made and still fall within the scope of the disclosure. For example, features described above in connection with one embodiment can be used with a different embodiment described herein and the combination still fall within the scope of the disclosure. It should be understood that various features and aspects of the disclosed embodiments can be combined with, or substituted for, one another in order to form varying modes of the embodiments of the disclosure. Thus, it is intended that the scope of the disclosure herein should not be limited by the particular embodiments described above. Accordingly, unless otherwise stated, or unless clearly incompatible, each embodiment of this invention may comprise, additional to its essential features described herein, one or more features as described herein from each other embodiment of the invention disclosed herein.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognise that the disclosure may be embodied or carried out in a manner that achieves one advantage or a group of advantages as taught herein without necessarily achieving other advantages as may be taught or suggested herein.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without other input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount.

Any methods disclosed herein need not be performed in the order recited. The methods disclosed herein include certain actions taken by a practitioner; however, they can also include any third-party instruction of those actions, either expressly or by implication. For example, actions such as “controlling a motor speed” include “instructing controlling of a motor speed.”

All of the methods and tasks described herein may be performed and fully automated by a computer system. The computer system may, in some cases, include multiple distinct computers or computing devices (e.g., physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (e.g., solid state storage devices, disk drives, etc.). The various functions disclosed herein may be embodied in such program instructions, and/or may be implemented in application-specific circuitry (e.g., ASICs or FPGAs) of the computer system. Where the computer system includes multiple computing devices, these devices may, but need not, be co-located. The results of the disclosed methods and tasks may be persistently stored by transforming physical storage devices, such as solid state memory chips and/or magnetic disks, into a different state. In some embodiments, the computer system may be a cloud-based computing system whose processing resources are shared by multiple distinct business entities or other users.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. A training system for a respiratory apparatus, the training system comprising:
a respiratory apparatus, and
a peripheral device which simulates one or more auxiliary device outputs and/or patient respiratory responses;
wherein the peripheral device communicates with the respiratory apparatus and provides the simulated one or more auxiliary device outputs and/or patient respiratory responses.

2. The training system of claim 1, wherein the peripheral device is one or more of a tablet, smartphone or personal computer.

3. The training system of claim 1 or 2, wherein the one or more auxiliary devices comprise a pulse oximeter device.

4. The training system of any of claims 1-3, wherein the peripheral device comprises software configured to model a virtual patient receiving therapy and provide feedback to the respiratory apparatus to simulate measurements of the virtual patient receiving therapy.

5. The training system of claim 4, wherein the virtual patient is configured to account for different oxygen requirements of different types of respiratory therapies.

6. The training system of claim 5, wherein the virtual patient is configured to account for the different oxygen requirements by adjusting one or more model parameters depending on a therapy mode configured on the respiratory apparatus.

7. The training system of claim 5 or 6, wherein the virtual patient is configured to model an SpO₂/FiO₂ ratio.

8. The training system of any of claims 4-7, wherein the virtual patient comprises a plurality of patient models of different ages that are related by linear factor(s).

9. The training system of claim 8, wherein the plurality of patient models comprise an adult model, a pediatric model, and a neonatal model.

10. The training system of claim 8 or 9, wherein a user interface of the peripheral device comprises one or more patient model choice elements configured to allow selection of a predefined patient model based on age.

11. The training system of any of claims 4-10, wherein the virtual patient is configured to account for different severity statuses.

12. The training system of claim 11, wherein a user interface of the peripheral device comprises one or more patient model choice elements configured to allow selection of a predefined severity status.

13. The training system of claim 11 or 12, wherein the severity statues comprise ICU, HDU, and regular ward.

14. The training system of any of claims 11-13, wherein the severity statues comprise a respiratory condition, ailment, disease, injury, and/or infection.

15. The training system of claim 14, wherein the respiratory condition, ailment, disease, injury, and/or infection comprises a stage I, II, III, or IV chronic obstructive pulmonary disease (COPD).

16. The training system of any of claims 1-15, wherein the respiratory apparatus is designed to administer high flow therapy.

17. The training system of any of claims 1-16, wherein the respiratory apparatus comprises a flow generator and connection to a supplementary gases supply.

18. The training system of any of claims 1-17, wherein the respiratory apparatus comprises a humidifier.

19. The training system of any of claims 1-18, wherein the simulated one or more auxiliary device outputs and/or patient respiratory responses comprise information representative of the virtual patient's SpO₂ data.

20. The training system of claim 19, wherein the virtual patient's SpO₂ data comprises plethysmography waveforms.

21. The training system of any of claims 1-20, wherein the simulated one or more auxiliary device outputs and/or patient respiratory responses comprise information representative of the virtual patient's respiratory rate.

22. The training system of any of claims 1-21, wherein the simulated one or more auxiliary device outputs and/or patient respiratory responses comprise information representative of the virtual patient's minute ventilation, tidal volume, and/or peak inspiratory demand.

23. The training system of any of claims 1-22, wherein the peripheral device is configured with further instructions to transmit supplementary gas signals that are representative of a supplementary gas being received into the respiratory apparatus.

24. The training system of any of claims 1-23, wherein the peripheral device is configured with further instructions to continuously transmit the simulated one or more auxiliary device outputs and/or patient respiratory responses and O₂ signals, wherein the simulated one or more auxiliary device outputs and/or patient respiratory responses are representative of the virtual patient's characteristics, and wherein the O₂ signals are representative of an O₂ supply.

25. A peripheral device which communicates with a respiratory apparatus to provide simulated outputs of at least one auxiliary device connectable to the respiratory apparatus, the peripheral device comprising:
a first communication interface, wherein the first communication interface electrically connects the peripheral device to an electrical or electronic communications port of the respiratory apparatus; and
a hardware processor operating software that provides instructions to a hardware processor to:
communicate simulated measurement information from the at least one auxiliary device to the electrical or electronic communications port of the respiratory apparatus.

26. The peripheral device of claim 25, wherein the peripheral device comprises software configured to model a virtual patient receiving therapy and communicate feedback to the respiratory apparatus in the form of the simulated measurement information of the virtual patient.

27. The peripheral device of claim 26, wherein the software further provides instructions to the hardware processor to adjust a virtual patient parameter configured to alter the simulated measurement information.

28. The peripheral device of any of claims 25-27, wherein the first communication interface is a connection via a USB port, a Bluetooth interface, or another wireless communication interface.

29. The peripheral device of any of claims 25-28, wherein the peripheral device is one or more of a tablet, smartphone or personal computer.

30. The peripheral device of any of claims 25-29, wherein the at least one auxiliary device comprises a pulse oximeter device.

31. The peripheral device of any of claims 25-30, wherein the simulated measurement information comprises information representative of the virtual patient's SpO₂ data.

32. The peripheral device of claim 31, wherein the virtual patient's SpO₂ data comprises plethysmography waveforms.

33. The peripheral device of any of claims 25-32, wherein the hardware processor is configured with further instructions to simulate the virtual patient such that the respiratory apparatus receives signals that are representative of the virtual patient's response.

34. The peripheral device of claim 33, wherein the signals that are representative of the virtual patient's response comprise signals representative of the virtual patient's respiratory rate.

35. The peripheral device of claim 33 or 34, wherein the signals that are representative of the virtual patient's response comprise signals representative of the virtual patient's minute ventilation, tidal volume, and/or peak inspiratory demand.

36. The peripheral device of any of claims 25-35, wherein the hardware processor is configured with further instructions to transmit supplementary gas signals that are representative of a supplementary gas being received into the respiratory apparatus.

37. The peripheral device of any of claims 25-36, wherein the hardware processor is configured with further instructions to continuously transmit virtual patient response signals and O₂ signals, wherein the virtual patient response signals are representative of the virtual patient's characteristics and wherein the O₂ signals are representative of an O₂ supply.

38. The peripheral device of any of claims 25-37, further comprising a second communication interface, wherein the second communication interface electrically connects the peripheral device to another auxiliary device.

39. The peripheral device of claim 38, wherein the second communication interface is a connection via a USB port, a Bluetooth interface, or another wireless communication interface.

40. The peripheral device of claim 38 or 39, wherein the another auxiliary device connected to the peripheral device via the second communication interface is an artificial lung.

41. A respiratory apparatus training system comprising:
a respiratory apparatus, including:
a valve configured to control an amount of supplementary gases entering the respiratory apparatus;
a flow generator configured to generate a flow of one or more gases, wherein the one or more gases comprises the supplementary gases or the respiratory apparatus is configured to simulate administration of the supplementary gases in response to one or more signals from a peripheral device simulating a virtual patient receiving therapy from the respiratory apparatus;
an apparatus user interface, wherein the respiratory apparatus is configured to receive operating parameters and/or operational settings based on input received via the apparatus user interface;
an apparatus communications interface configured to receive and transmit the one or more signals from the peripheral device; and
an apparatus controller, wherein the apparatus controller is configured to control the flow generator and the valve and transmit signals to the apparatus user interface, wherein the respiratory apparatus is configured to operate based on the received operating parameters and/or operational settings and the one or more signals from the peripheral device; and
the peripheral device, including:
a peripheral device controller;
a peripheral device user interface; and
a peripheral device communications interface to receive and transmit the one or more signals to the respiratory apparatus,
wherein the one or more signals to the respiratory apparatus further indicate a plurality of parameters of the virtual patient in response to therapy administered by the respiratory therapy device, the peripheral device user interface configured to allow the plurality of parameters and/or characteristics of the virtual patients to be modified.

42. The respiratory apparatus training system of claim 41, wherein the supplementary gases comprise oxygen.

43. The respiratory apparatus training system of claim 42, wherein the respiratory apparatus is configured to adjust a FdO₂ value of the flow of one or more gases.

44. The respiratory apparatus training system of claim 43, wherein the apparatus controller is configured to control the valve so as to adjust the FdO₂ value.

45. The respiratory apparatus training system of claim 43 or 44, wherein the apparatus controller is configured to adjust the FdO₂ value in response to the one or more signals from the peripheral device comprising a selection of a high pressure oxygen source.

46. The respiratory apparatus training system of any of claims 41-45, wherein the plurality of parameters comprises the virtual patient's SpO₂.

47. The respiratory apparatus training system of any of claims 41-46, wherein the plurality of parameters comprises a supplementary gases source.

48. The respiratory apparatus training system of any of claims 41-47, wherein the characteristics of the virtual patient comprise age, severity status, and/or ward type.

49. The respiratory apparatus training system of any of claims 41-48, wherein settings of the peripheral device are configured to be modified by a user to change the one or more signals.

50. The respiratory apparatus training system of any of claims 41-49, wherein the training system is used for training an operator of the respiratory apparatus.

51. The respiratory apparatus training system of any of claims 41-50, wherein the respiratory apparatus further comprises a supplementary gas source port in communication with the valve.

52. The respiratory apparatus training system of any of claims 41-51, wherein the peripheral device further comprises a non-volatile memory.

53. A training system for training operators in the use of a respiratory support device comprising:
a respiratory apparatus, including:
a flow generator configured to generate a flow of one or more gases;
an apparatus user interface, wherein the respiratory apparatus is configured to receive operating parameters and/or operational settings based on inputs received at the apparatus user interface;
an apparatus communications interface to receive from and transmit signals to another device in electrical communication with the respiratory apparatus; and
a peripheral device, including:
a peripheral device controller;
a peripheral device user interface; and
a peripheral device communications interface to receive from and transmit signals to another device in electrical communication with the peripheral device,
wherein the peripheral device is configured to communicate with the respiratory apparatus to transmit simulated signals including a signal representative of a supplementary gases and a signal representative of a virtual patient response;
wherein the training system is configured to allow an operator to learn operation of the respiratory apparatus based on the simulated signals from the peripheral device.

54. The training system of claim 53, wherein the peripheral device user interface is configured to allow the operator to modify the signal representative of the supplementary gases and/or the signal representative of the virtual patient response.

55. The training system of claim 53 or 54, wherein the apparatus user interface is configured to allow the operator to change the operating parameters and/or operational settings.

56. The training system of any of claims 53-55, wherein the respiratory apparatus is configured to have a training mode, wherein the apparatus user interface is configured to allow the operator to enter the training mode, wherein the training mode allows the respiratory apparatus to receive the simulated signals from the peripheral device and execute the operational parameters and/or operational settings that are set by the operator.

57. The training system of any of claims 53-56, wherein the respiratory apparatus is a high flow apparatus that provides high flow therapy.

58. The training system of any of claims 53-57, wherein the respiratory apparatus further comprises a supplementary gases source port, a valve in communication with the supplementary gases port to control an amount of supplementary gases entering the respiratory apparatus.

59. The training system of any of claims 53-58, wherein the respiratory apparatus further comprises a humidifier and a heated breathing tube.
